(19) United States
(12) Patent Application Publication
(10) Pub. No.: US 2025/0361504 A1
(43) Pub. Date: Nov. 27, 2025

(54) SPATIAL MRNA/PROTEIN CO-ASSAYS USING APTAMERS

(71) Applicant: ILLUMINA, INC., San Diego, CA (US)

(72) Inventors: Mats EKSTRAND, San Diego, CA (US); Andrew SLATTER, Cambridge (GB); Andrea MANZO, San Diego, CA (US); Lena STORMS, San Diego, CA (US); Maria Martins VITORIANO, Baldock (GB)

(21) Appl. No.: 18/872,127

(22) PCT Filed: Dec. 22, 2023
(86) PCT No.: PCT/US2023/085774

§ 371 Date: Dec. 5, 2024

Related U.S. Application Data
(60) Provisional application No. 63/477,096, filed on Dec. 23, 2022.

(51) Int. Cl. C12N 15/10; C12N 15/115; C12Q 1/6806; C12Q 1/6809; C12Q 1/6823; C12Q 1/6844; G01N 21/64; G01N 33/68

(52) U.S. Cl. CPC C12N 15/1065; C12N 15/115; C12Q 1/6806; C12Q 1/6809; C12Q 1/6823; C12Q 1/6844; G01N 21/6456; G01N 33/6896; C12N 2310/16; G01N 2333/705; G01N 2333/914; G01N 2800/2821

(57) ABSTRACT

The present disclosure relates, in general, to methods of preparing a spatial proteome and/or transcriptome sequencing library. The spatial proteome and/or transcriptome sequencing library from a biological sample is useful, in some aspects, to determine a genetic profile and help diagnose a subject who has or is at risk of having a disorder, and improve treatment of the subject.

### CROSS REFERENCE TO RELATED APPLICATIONS

The present application claims the benefit of U.S. Provisional Application No. 63/477,096, filed Dec. 23, 2022, which is incorporated herein by reference in its entirety.

### FIELD

The present disclosure is generally related to methods of preparing spatial proteogenomic sequencing libraries.

### SUMMARY

There is a need for technologies to map both spatial transcriptomes and spatial proteomes in the same tissue slice. Co-localization of protein and mRNA signals will lead to a better understanding of how mRNA and protein expression are co-regulated. Certain diseases (for example and without limitation, Alzheimer's disease) are characterized by aberrant protein deposition, and understanding how gene expression is altered near these protein deposits may elucidate the mechanisms underlying these disorders. Aptamers targeting cell membrane or nuclear membrane proteins can also be used to define cell and nuclear boundaries, simplifying cell segmentation of spatial transcriptomic data.

Commercially available ex situ spatial assays that can detect both proteins and mRNA are limited to detecting only a few proteins using immunofluorescence. Future products that can detect more proteins are based on oligo-conjugated antibodies. However, aptamers can also be used to detect proteins in situ, and have several advantages over antibodies. Aptamers are oligonucleotides (e.g., DNA or RNA oligonucleotides) that can specifically bind proteins. Because of their small size compared to antibodies, aptamers can diffuse more readily into tissue. Aptamers are more stable and less sensitive to temperature and pH changes. Aptamers can also be manufactured more reproducibly and at higher scale compared to antibodies.

Aptamers can be modified with a sequence to enable capture on a barcoded surface. However, there are a few challenges in designing a spatial mRNA/protein co-assay:
1. Addition of a long sequence to the aptamer to enable capture can impact protein binding by the aptamer.
2. Aptamers are difficult to remove from their target proteins.
3. Free aptamers that are not bound to protein must be prevented from binding to the barcoded surface
4. Proteins are much more abundant than mRNA molecules, and protein levels span a large dynamic range.

In various aspects, the present disclosure provides methods for using aptamers in a spatial mRNA/protein co-assay that address these challenges.

In some aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a cleavage site; (c) removing aptamers in the plurality of aptamers that did not associate with a protein in the biological sample; (d) cleaving the plurality of aptamers to release (i) the target nucleotide sequence and (ii) the aptamer barcode nucleotide sequence, thereby resulting in association of the target nucleotide sequence with the capture nucleotide sequence, and thereby preparing the spatial proteome sequencing library. In some aspects, the UMI comprises a spatial barcode nucleotide sequence and is included in the aptamer and not the capture nucleotide sequence. In some aspects, the surface further comprises a blocker nucleic acid that is hybridized to at least a portion of the capture nucleotide sequence. In some aspects, the blocker nucleic acid is removed from the capture oligonucleotide after step (c). In various aspects, the plurality of aptamers is cleaved via ultraviolet radiation, an enzyme, or chemical cleavage. In some aspects, methods of the disclosure further comprise (e) extending the capture nucleotide sequence to create copies of the individual aptamers, thereby creating extended capture oligonucleotides. In some aspects, methods of the disclosure further comprise (f) adding a template switch oligonucleotide (TSO) to the 3′ end of the extended capture oligonucleotides.

In some aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a truncated adapter nucleotide sequence; (c) removing aptamers in the plurality of aptamers that did not associate with a protein in the biological sample; (d) eluting the individual aptamers from the individual proteins, thereby resulting in association of the target nucleotide sequence with the capture nucleotide sequence, and thereby preparing the spatial proteome sequencing library. In some aspects, the surface further comprises a blocker nucleic acid that is hybridized to at least a portion of the capture nucleotide sequence. In further aspects, the blocker nucleic acid is removed from the capture oligonucleotide after step (c). In some aspects, methods of the disclosure further comprise (e) extending the capture nucleotide sequence to create copies of the individual aptamers, thereby creating extended capture oligonucleotides. In some aspects, methods of the disclosure further comprise (f) hybridizing the truncated adapter nucleotide sequence to a full length adapter nucleotide sequence primer and extending to synthesize a second strand.

In further aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamer complexes in the plurality of aptamer complexes with individual proteins in the biological sample, wherein each aptamer complex in the plurality of aptamer complexes comprises: (1) an aptamer comprising (i) the capture nucleotide sequence; and (ii) an aptamer-specific nucleotide sequence; and (2) an oligonucleotide hybridized to the aptamer prior to the contacting, the oligonucleotide comprising (i) the target nucleotide sequence; (ii) a sequence complementary to the aptamer-specific nucleotide sequence; and (iii) an aptamer barcode nucleotide sequence, wherein after the association of individual aptamer complexes in the plurality of aptamer complexes with individual proteins in the biological sample, the oligonucleotide is released from the aptamer thereby resulting in association of the target nucleotide sequence of the released oligonucleotide with the capture nucleotide sequence of a capture oligonucleotide of the plurality of capture oligonucleotides; thereby preparing the spatial proteome sequencing library. In some aspects, the aptamer-specific nucleotide sequence is about 5 to about 20 nucleotides in length. In further aspects, the aptamer-specific nucleotide sequence is about 10 nucleotides in length. In some aspects, the surface further comprises a blocker nucleic acid that is hybridized to at least a portion of the capture nucleotide sequence. In further aspects, the blocker nucleic acid is removed from the capture oligonucleotide after the contacting. In some aspects, the association of individual aptamer complexes in the plurality of aptamer complexes with individual proteins in the biological sample results in release of the oligonucleotide from the aptamer. In some aspects, after the association of individual aptamer complexes in the plurality of aptamer complexes with individual proteins in the biological sample, a condition is changed thereby resulting in release of the oligonucleotide from the aptamer. In further aspects, the condition is temperature, pH, or salt concentration. In still further aspects, after the association of individual aptamer complexes in the plurality of aptamer complexes with individual proteins in the biological sample, formamide is added thereby resulting in release of the oligonucleotide from the aptamer. In various aspects, the blocker oligonucleotide is removed from the capture oligonucleotide by exonuclease digestion. In further aspects, the exonuclease digestion is performed using T7 exonuclease or lambda exonuclease. In some aspects, the aptamers comprise a detectable moiety. In some aspects, the detectable moiety is a fluorescent moiety.

In further aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; and (ii) an aptamer barcode nucleotide sequence; (c) removing aptamers in the plurality of aptamers that did not associate with a protein in the biological sample; (d) eluting the individual aptamers from the individual proteins, thereby resulting in hybridization of the target nucleotide sequence with the capture nucleotide sequence, and thereby preparing the spatial proteome sequencing library. In some aspects, the plurality of capture oligonucleotides comprises a cleavable site at the 5′ end. In some aspects, step (d) further comprises contacting at least one aptamer of the plurality of aptamers with a blocker nucleic acid, thereby forming a blocked aptamer, wherein the blocker nucleic acid is complementary to the target nucleotide sequence, and wherein the blocked aptamer is unable to associate with the capture nucleotide sequence. In some aspects, the surface further comprises a blocker nucleic acid that is hybridized to at least a portion of the capture nucleotide sequence. In some aspects, the blocker nucleic acid is removed from the capture oligonucleotide after step (c). In some aspects, the eluting in step (d) comprises digesting the proteins in the biological sample or competition with excess aptamers. In some aspects, the method further comprises (e) extending the capture nucleotide sequence to create copies of the individual aptamers, thereby creating extended capture oligonucleotides. In some aspects, step (e) further comprises hybridizing a plurality of aptamer barcoded oligonucleotides to the extended capture oligonucleotides, and extending the extended capture oligonucleotides, thereby creating a plurality of barcoded capture oligonucleotides, wherein each of the aptamer barcoded oligonucleotides comprises at least a portion of an individual aptamer sequence. In some aspects, the plurality of aptamer barcoded oligonucleotides comprise a plurality of aptamer blocker nucleic acids, wherein each of the aptamer blocker nucleic acids comprises at least a portion of an individual aptamer sequence.

In some aspects, the method further comprises contacting a second plurality of aptamers to the biological sample on the surface, the contacting resulting in association of individual aptamers in the second plurality of aptamers with individual proteins in the biological sample, wherein each aptamer in the second plurality of aptamers comprises a detectable moiety. In some aspects, the detectable moiety is a fluorophore. In some aspects, each aptamer in the second plurality of aptamers comprises the target nucleotide sequence and a truncated adapter nucleotide sequence. In some aspects, each aptamer in the second plurality of aptamers further comprises an aptamer barcode nucleotide sequence. In some aspects, after contacting the second plurality of aptamers to the biological sample on the surface, the method further comprises imaging the biological sample, thereby obtaining an image of the biological sample. In some aspects, the method does not comprise contacting the biological sample with hematoxylin and eosin (H&E) staining reagents. In some aspects, at least one aptamer in the second plurality of aptamers is specific for a cell membrane-associated protein. In some aspects, at least one aptamer in the second plurality of aptamers is specific for a nuclear membrane-associated protein. In some aspects, at least one aptamer in the second plurality of aptamers is specific for a cell membrane-associated protein and at least one aptamer in the second plurality of aptamers is specific for a nuclear membrane-associated protein, and wherein the at least one aptamer specific for the nuclear membrane-associated protein comprises a different detectable moiety than the at least one aptamer specific for the cell membrane-associated protein. In some aspects, the at least one aptamer specific for the nuclear membrane-associated protein comprises a different aptamer barcode nucleotide sequence than the at least one aptamer specific for the cell membrane-associated protein. In some aspects, the cell membrane associated protein is E-cadherin, N-cadherin, or a Na⁺/K⁺-ATPase. In some aspects, the nuclear membrane-associated protein is a nuclear pore complex protein.

In any of the aspects or aspects of the disclosure, the biological sample is from a mammal. In further aspects, the biological sample is from a human.

In further aspects, the disclosure provides a method of identifying a disorder in a subject having or at risk of having the disorder comprising: i) generating a spatial proteomic and/or transcriptomic library from a biological sample from the subject according to the methods of the disclosure, ii) comparing proteomic and/or genetic information from the sample proteomic and/or transcriptomic library to a control proteomic and/or transcriptomic library, iii) identifying a genetic variation in the sample proteomic and/or transcriptomic library associated with the disease. In some aspects, the disorder is a neurodegenerative disorder. In further aspects, the disorder is Alzheimer's disease.

### BRIEF DESCRIPTION OF THE DRAWINGS

FIG. 1 shows a spatial proteomics workflow using aptamers tagged with a polyA sequence at the 3′ end and a truncated B15 adapter at the 5′ end.

FIG. 2 shows how methods provided herein can be modified to be compatible with a spatial protein/mRNA co-assay.

FIG. 3 shows an exemplary variation of a workflow of the disclosure using aptamers that contain cleavable tags.

FIG. 4 shows an exemplary variation of a workflow of the disclosure using tagged aptamers bound to single-stranded DNA, wherein single-stranded DNA is released from tagged aptamers upon protein binding.

FIG. 5 depicts exemplary methods provided by the disclosure that address the problem of accommodating a large dynamic range and copy number of proteins.

FIGS. 6A-6F show an exemplary variation of a workflow of the disclosure using modified aptamers including a unique barcode and an adapter sequence, wherein blocker oligonucleotides complementary for the aptamer adapter sequence are included to accommodate a large dynamic range of protein abundance. FIG. 6A is a representative illustration of the cellular milieu including a mixure of proteins (labeled as “1”, “2”, and “X” for mRNA binding protein) and mRNA. FIG. 6B shows a step of permeabilizing the cell to allow the modified aptamers into the cells. A wash step (not show) removes unbound aptamers. FIG. 6C shows immobilized surface primers hybridized to blocking oligonucleotides. These surface primers can have the same or different capture sequences. FIG. 6D shows a step of deblocking the surface primers to expose a free 3′ end. FIG. 6E shows a step of digesting the proteins to release the mRNA and aptamers, and, optionally, adding dynamic range compression (DRC) blocker oligonucleotides specific for abundant protein aptamers. FIG. 6F shows a step of capturing the released mRNA and aptamer barcodes on the immobilized surface primers followed by reverse transcription and polymerase extension to generate covalently attached complements of the mRNA and aptamer barcodes. In some aspects, the mRNA is captured on the surface after permeabilization in the presence of aptamer to minimize the loss of cellular mRNA during processing. In some aspects, the mRNA capture sequences are unblocked and the aptamer capture sequences are blocked.

FIGS. 7A-7D show an exemplary variation of a workflow of the disclosure using DRC blockers to maintain abundant SOMAmer(s) in solution. FIG. 7A shows, following cellular permeabilization, aptamer binding, and protein digestion, the copying by reverse transcription and polymerase extension of released mRNA and SOMAmers on the immobilized surface primers. STUBBY corresponds to a universal adapter sequence at the 3′ end of the aptamer (i.e., the SOMAmer). The addition of a DRC blocker to the SOMAmer (i.e., a blocker oligo including a complement to the STUBBY sequence and a complement to the SOMAmer sequence. FIG. 7B shows a step of, following reverse transcription and polymerase extension, washing away unbound mRNA and SOMAmers, leaving behind immobilized, extended surface primers including cDNA of the mRNA or complements of the STUBBY and SOMAmer sequences. FIG. 7C shows a step of adding a SOMAmer barcode to the extended surface primers including the complement of the SOMAmer sequence, wherein an oligo including a SOMAmer-SEQ sequence, barcode sequence, and B-15′ sequencing primer sequence is hybridized to the extended surface primer and copied. A “dummy” oligo is included which lack a barcode sequence and sequencing primer sequence, and may acts as a DRC blocker for highly abundant SOMAmers. FIG. 7D shows a cleavage step to release the extended surface primers included the SOMAmer barcode and B-15 adapter sequence, followed by library prep steps, including addition of sequencing adapters using PCR. Sequencing the barcode for the SOMAmer provides alternative way to identify the SOMAmer sequence without directly sequencing it.

FIGS. 8A-8B show an exemplary variation of workflow of the disclosure using fluorescently-labeled aptamers targeting cell membranes, for examples, for cell segmentation analysis and image alignment processing. FIG. 8A shows the steps of adding the fluorescently-labeled aptamers to proteins, flowing away any unbound aptamers, and then imaging the tissue to visualize the bound aptamers. The immobilized surface primers on the solid support are blocked with, for example, complementary blocking oligonucleotides. FIG. 8B shows the steps of removing the blocking oligo from the surface primers with, for example, T7 or lambda exonuclease, eluting the protein-bound aptamers with excess unlabeled aptamers, capturing the eluted aptamers, extending the surface primers to copy the aptamer to the surface, and performing second-strand synthesis with a full B15 adapter sequence oligo hybridized to the truncated B15 adapter.

FIGS. 9A-9B show various examples of cell-membrane targeted aptamers and nuclear-membrane targeting aptamers. FIG. 9A shows multiple types of cell-membrane targeted aptamers containing a common fluorophore and barcode to, for example, enable membrane detections across different tissue types. Each aptamer may contain a cell membrane barcode and a truncated B15 adapter sequence. FIG. 9B shows exemplary aptamers for the co-detection of cell membrane and nuclear membrane proteins using differentially labeled aptamers (e.g., aptamers with different fluorophores and barcode sequences).

### DETAILED DESCRIPTION

The emerging field of spatial proteogenomics is being driven by the development of new technologies that allow the mapping of single cell-omes to their spatial locations in a tissue slice. One method for spatially mapping single-cell transcriptomes (called the ex situ approach) involves the use of a surface coated with barcoded oligonucleotides, where the spatial location of each barcode is known. The barcoded oligonucleotides are localized into individual features, where every oligonucleotide in the same feature carries the same spatial barcode. Different implementations of this surface include a bead array, a spotted array, a clustered flow cell, or clustered particles arranged on a surface. These oligonucleotides also contain an oligo (dT) capture sequence that binds mRNA and acts as a primer for reverse transcription. A tissue section is then placed on this surface and polyA mRNA molecules within the tissue diffuse to the features and are captured on the surface. The captured RNA is reverse transcribed into cDNA, linking the spatial barcode with the cDNA sequence. This is followed by library prep and sequencing on a standard (e.g., Illumina) sequencer. During analysis, the spatial barcode is used to map the physical location of the molecule from which the read is derived.

### Terms

As used in this specification and the enumerated paragraphs herein, the singular forms “a,” “an,” and “the” include plural reference unless the context clearly dictates otherwise.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

As used herein, the terms “includes,” “including,” “includes,” “including,” “contains,” “containing,” “have,” “having,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter. Similarly, “comprise,” “comprises,” “comprising” “include,” “includes,” and “including” are interchangeable and not intended to be limiting.

As used herein, “surface” can refer to a part of a substrate or support structure that is accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fibre; metal; inorganic glass; optical fibre bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl)acrylamide-coacrylamide) (PAZAM, see, for example, U.S. Pat. App. Pub. No. 2014/0079923 A1, which is incorporated herein by reference). In some examples, a support structure can include one or more layers. Non-limiting examples of a surface include a bead array, a spotted array, clustered particles arranged on a surface of a chip, a film, a multi-well plate, and a flow cell.

In a certain aspect, a “surface” and/or “substrate” disclosed herein may further comprise islands or clusters of immobilized capture agents or capture oligonucleotides. The islands or clusters can be generated on the surface of a substrate (e.g., a flowcell) by using bridge amplification. In such a case, the substrate comprises a plurality of immobilized capture oligonucleotides on the surface of the substrate, which bind with complementary adapter regions present on nearby primers or oligonucleotides to form bridge-like structures; these bridge-like structures are then extended using a polymerase enzyme, generating a double stranded molecule, that is then denatured to leave a single-stranded capture oligo anchored to the substrate. After multiple iterations of the foregoing process, islands or clusters of immobilized capture oligonucleotides are created. An example of the foregoing process that can be used with the methods and compositions disclosed herein can be found in WO 2022/015913 A1, which is incorporated herein by reference in its entirety. In a particular aspect, the nearby primers or oligonucleotides are attached to the substrate (e.g., a flowcell) by a selectively cleavable linker. Each island or cluster may be roughly circular or oval in shape. Each island or cluster may have an average diameter of 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1200 nm, or a range that includes or is in between any two of the forgoing diameters. In a further aspect, the surface of the substrate (e.g., a flowcell) comprises per 1 mm² of surface area 0.3, 0.4, 0.5, 0.6. 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, or 2.5 million clusters, or range including or between any two of the forgoing numbers. In a particular aspect, a “substrate” as disclosed herein comprises islands or clusters of immobilized capture oligonucleotides comprising adapter sequence(s), a spatial address sequence, an optional sequence primer site, and a capture moiety for a targeted analyte. In yet a further aspect, each cluster or island on the substrate (e.g., a flowcell) comprises capture oligonucleotides that have a unique spatial address sequence, so the x,y location of each cluster or island can be identified. In such a case, the x,y location of each cluster or island can be determined by decoding the spatial address sequence. Methods to decode the spatial address sequence include, but are not limited, the decoding-by-hybridization or the decoding-by-sequencing methods disclosed herein.

As used herein, the term “interstitial region” refers to an area in a substrate or on a surface that separates other areas of the substrate or surface. For example, an interstitial region can separate one feature of an array from another feature of the array. The two regions that are separated from each other can be discrete, lacking contact with each other. In another example, an interstitial region can separate a first portion of a feature from a second portion of a feature. The separation provided by an interstitial region can be partial or full separation. Interstitial regions will typically have a surface material that differs from the surface material of the features on the surface. For example, features of an array can have an amount or concentration of capture agents or capture oligonucleotides that exceeds the amount or concentration present at the interstitial regions. In some aspects the capture agents or primers may not be present at the interstitial regions.

In some aspects, the substrate includes an array of wells or depressions in a surface. This may be fabricated as is generally known in the art using a variety of techniques, including, but not limited to, photolithography, stamping techniques, molding techniques and micro-etching techniques. As will be appreciated by those in the art, the technique used will depend on the composition and shape of the array substrate.

Exemplary flow cells include, but are not limited to those used in a nucleic acid sequencing apparatus such as flow cells for the Genome Analyzer®, MiSeq®, NextSeq® or HiSeq® platforms commercialized by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference. A flowcell can be “a nonpattemed flowcell”, where the surface(s) of the flowcell comprises randomly or semi-randomly arranged features (e.g., areas comprising clusters or islands of oligonucleotides). Alternatively, the flowcell can be a “patterned flowcell,” where the flowcell comprises features (e.g., nanowells) at fixed locations across the surface(s) of the flowcell. The features of a “patterned flowcell” can further comprise immobilized oligonucleotides, or clusters or islands of immobilized oligonucleotides A “patterned flowcell” can be an “ordered substrate” in that the features of the patterned flowcell have an assigned x,y spatial address, or an x,y spatial address that can be readily determined.

By “complementary” is meant that an oligonucleotide comprises a sequence of nucleotides that can form a double-stranded structure by matching base-pairs with another oligonucleotide or part thereof. By “complementary” is meant that the oligonucleotide has at least 85%, 90%, 95%, 98%, 99% or 100% overall sequence identity to the complementary sequence.

In any of the aspects or aspects of the disclosure, methods described herein comprise a sequencing procedure, for example and without limitation a sequencing-by-synthesis (SBS) technique or nanopore sequencing. Briefly, SBS can be initiated by contacting the barcodes with one or more labeled nucleotides, DNA polymerase, etc. Those features where a primer is extended using the sequences comprising the barcode as a template will incorporate a labeled nucleotide that can be detected. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for aspects that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a library produced by the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference.

As used herein, a “primer” is a nucleic acid molecule that can hybridize to a target sequence, such as an adapter attached to a library fragment. In some aspects, an amplification primer can serve as a starting point for template amplification and cluster generation. As another example, a synthesized nucleic acid (template) strand may include a site to which a primer (e.g., a sequencing primer) can hybridize in order to prime synthesis of a new strand that is complementary to the synthesized nucleic acid strand. Any primer can include any combination of nucleotides or analogs thereof. In some examples, the primer is a single-stranded oligonucleotide or polynucleotide. The primer length can be any number of bases long and can include a variety of non-natural nucleotides. In various aspects, the sequencing primer is a short strand, ranging from 5 to 60 bases, from 10 to 60 bases, from 10 to 20 bases, from 10 to 30 bases, from 10 to 40 bases, from 10 to 50 bases, or from 20 to 40 bases. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an aspect the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another aspect the primer is an RNA primer. In aspects, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “unique molecular identifier” or “UMI” refers to a molecular tag, either random, non-random, or semi-random, that may be attached to a nucleic acid. When incorporated into a nucleic acid, a UMI can be used to correct for subsequent amplification bias by directly counting unique molecular identifiers (UMIs) that are sequenced after amplification. A UMI can be attached to similar nucleic acids, e.g., adapters, making each nucleic acid unique. In some aspects, the UMI comprises a spatial barcode.

As used herein, a “semi-random” nucleotide sequence comprises or consists of a partially pre-determined nucleotide sequence combined with a random nucleotide sequence.

As used herein, the term “adapter” refers generally to any linear nucleic acid molecule that can be added to an oligonucleotide of the disclosure. In some aspects, adapters are copied onto the library molecules using templated polymerase synthesis. In some aspects, adapters include two reverse complementary oligonucleotides forming a double-stranded structure. In some aspects, an adapter includes two oligonucleotides that are complementary at one portion and mismatched at another portion, forming a Y-shape or fork-shaped adapter that is double stranded at the complementary portion and has two floppy overhangs at the mismatched portion. In some aspects, an adapter is a template switch oligonucleotide (TSO) adapter.

The term “template switch oligonucleotide” refers to an oligonucleotide template to which polymerase activity is switched from an initial template (e.g., a single-stranded nucleic acid provided by a sample of the invention). In one aspect of the invention, the template switch oligonucleotide is a DNA/RNA hybrid oligonucleotide that is used by a template-dependent DNA or RNA polymerase (preferably RT, preferably MMLV RT) to continue reverse transcription, i.e., template-independent, after the enzyme (preferably MMLV RT) reaches the 5'-end of the template nucleic acid and adds nucleotides to the 3'-end of the synthesized cDNA or cRNA strand by its terminal transferase activity. The 3'-end of the TSO hybridizes to nucleotides added by the terminal transferase activity of the template-dependent DNA or RNA polymerase, effectively extending the 5'-end of the template DNA or RNA, such that the template-dependent DNA or RNA polymerase (preferably RT, more preferably MMLV RT) also reverse transcribes the remaining 5'-portion of the TSO, which contains the defined sequence to be added to the 5'-end of the template nucleic acid. The TSO may comprise one or more modified or non-naturally occurring nucleotides (or analogs thereof). For example, the template switching oligonucleotide may comprise one or more nucleotide analogs (e.g., LNA, FANA, 2'-O-methyl ribonucleotide, 2'-fluoro ribonucleotide, etc.), ligation modifications (e.g., phosphorothioate, 3′-3′ and 5′-5′ reverse ligation), 5' and/or 3' terminal modifications (e.g., 5' and/or 3' amino, biotin, DIG, phosphate, thiol, dye, quencher, etc.), one or more fluorescently labeled nucleotides, or any other feature that provides a desired function to the template switching oligonucleotide.

The terms “P5” and “P7” may be used when referring to examples of adapters. The terms “P5′” (P5 primer) and “P7′” (P7 primer) refer to the complement of P5 and P7, respectively. It will be understood that any suitable adapter can be used in the methods presented herein, and that the use of P5 and P7 are exemplary aspects only. Uses of adapters such as P5 and P7 or their complements on flowcells are known in the art, as exemplified by the disclosures of WO 2007/010251, WO 2006/064199, WO 2005/065814, WO 2015/106941, WO 1998/044151, and WO 2000/018957, each of which is incorporated herein by reference in its entirety. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the term “barcode” is intended to mean a series of nucleotides in an oligonucleotide that can be used to identify the oligonucleotide, a spatial address on a surface (i.e., a “spatial barcode” or “spatial address sequence”), a characteristic of the oligonucleotide, and/or a manipulation that has been carried out on the oligonucleotide. The barcode can be a naturally occurring nucleotide sequence or a nucleotide sequence that does not occur naturally in the organism from which the barcoded nucleic acid was obtained. In aspects, a barcode is unique in a pool of barcodes that differ from one another in sequence, or is uniquely associated with a particular sample polynucleotide in a pool of sample polynucleotides. In aspects, every barcode in a pool of adapters is unique, such that sequencing reads including the barcode can be identified as originating from a single sample polynucleotide molecule on the basis of the barcode alone. In other aspects, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In aspects, barcodes are about or at least about 5, 6, 7, 8, 9, 10, 15, 20, 25, 30, 40, 50, 75 or more nucleotides in length. In aspects, barcodes are shorter than 20, 15, 10, 9, 8, 7, 6, or 5 nucleotides in length. In aspects, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In aspects, each barcode in a plurality of barcodes differs from every other barcode in the plurality by at least three nucleotide positions, such as at least 3, 4, 5, 6, 7, 8, 9, 10, or more nucleotide positions. In some aspects, substantially degenerate barcodes may be known as random. In some aspects, a barcode may include a nucleic acid sequence from within a pool of known sequences. In some aspects, the barcodes may be pre-defined.

As used herein, a “biological sample” may include one or more biological or chemical substances, such as nucleic acids, oligonucleotides, proteins, cells, tissues, organisms, and/or biologically active chemical compound(s), such as analogs or mimetics of the aforementioned species. In some instances, the biological sample may include whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, viruses including viral pathogens, liquids containing multi-celled organisms, biological swabs and biological washes. In further examples, the sample can be derived from an organ, including for example, an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; an organ of the central nervous system such as brain, brainstem, cerebellum, spinal cord, cranial nerve, or spinal nerve; a sensory organ such as eye, ear, nose, or tongue; or an organ of the integument such as skin, subcutaneous tissue or mammary gland. In various aspects, the tissue can be derived from a multicellular organism. In some aspects, a tissue section can be contacted with a surface, for example, by laying the tissue on the surface. The tissue can be freshly excised from an organism or it may have been previously preserved for example by freezing (e.g., fresh frozen tissue), embedding in a material such as paraffin (e.g., formalin fixed paraffin embedded (FFPE) samples), formalin fixation, infiltration, dehydration or the like. Optionally, a tissue section can be attached to a surface, for example, using techniques and compositions described in, for example, U.S. Pat. No. 11,390,912, incorporated by reference herein in its entirety. In some aspects, a tissue can be permeabilized and the cells of the tissue lysed when the tissue is in contact with a surface. Any of a variety of treatments can be used such as those set forth above in regard to lysing cells. Target proteins and/or nucleic acids that are released from a tissue that is permeabilized can be captured by capture oligonucleotides on the surface. The thickness of a tissue sample or other biological sample that is contacted with a surface in a method set forth herein can be any suitable thickness desired. In representative aspects, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a biological sample that is contacted with a surface will be no more than 100 μm, 50 μm, 10 μm, 5 μm, 1 μm, 0.5 μm, 0.25 μm, 0.1 μm or thinner.

As used herein, the term “permeable” refers to a property of a substance that allows certain materials to pass through the substance. “Permeable” may be used to describe a biological sample, such as a cell or nucleus, in which analytes in the biological sample can leave the biological sample. “Permeabilize” is an action taken to cause, for example, a biological sample (e.g., a cell) to release its analytes. In some examples, permeabilization of a biological sample is accomplished by affecting the integrity (e.g., compromising) of a biological sample membrane (e.g., a cellular or nuclear membrane) such as by application of a protease or other enzyme capable of disturbing a membrane allowing analytes to diffuse out of the biological sample. In some aspects, permeabilizing a biological sample does not release the biomolecules (e.g., proteins and/or nucleic acids) contained within the sample.

As used herein, a “capture oligonucleotide” is generally an oligonucleotide comprising a nucleotide sequence capable of hybridizing or otherwise associating with an aptamer or other oligonucleotide as described herein (e.g., a mRNA, a single-stranded oligonucleotide released from an aptamer complex following association of the aptamer complex with a protein, a probe binding to an mRNA target). The nucleotide sequence capable of hybridizing or otherwise associating with an aptamer or other oligonucleotide is, for example and without limitation, a universal sequence (e.g., a polyT sequence), or a target-specific sequence. A capture oligonucleotide can comprise additional elements, including but not limited to a unique molecular identifier (UMI), a spatial barcode, primer sequences to amplify from (e.g., A14-ME), sequences that are used to generate the barcoded features (e.g., a P7 sequence used in clustering and a SBS12 sequence used as the sequencing primer binding site) or a combination thereof.

A “universal sequence” as used herein refers to a common nucleotide sequence among a plurality of capture oligonucleotides. A common nucleotide sequence can be, for example, a sequence complementary to the same adapter sequence. Universal capture oligonucleotides are applicable for interrogating a plurality of different oligonucleotides without necessarily distinguishing the different species whereas target-specific capture sequences are applicable for distinguishing the different species. A non-limiting example of a universal sequence is a polyT nucleotide sequence.

As used herein, “hybridize” is intended to mean noncovalently associating a first oligonucleotide to a second oligonucleotide along the lengths of those polymers to form a double-stranded “duplex.” For instance, two DNA oligonucleotide strands may associate through complementary base pairing. The strength of the association between the first and second oligonucleotides increases with the complementarity between the sequences of nucleotides within those oligonucleotides. The strength of hybridization between oligonucleotides may be characterized by a temperature of melting (Tₘ) at which 50% of the duplexes have oligonucleotide strands that disassociate from one another. Oligonucleotides that are “partially” hybridized to one another means that they have sequences that are complementary to one another, but such sequences are hybridized with one another along only a portion of their lengths to form a partial duplex. Oligonucleotides with an “inability” to hybridize include those that are physically separated from one another such that an insufficient number of their bases may contact one another in a manner so as to hybridize with one another. For example, hybridization can be performed at a temperature ranging from 15° C. to 95° C. In some aspects, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other aspects, the stringency of the hybridization can be further altered by the addition or removal of components of the buffered solution.

As used herein, the term “plurality” is intended to mean a population of two or more members, which may all be the same or two or more members may be different. Pluralities may range in size from small, medium, large, to very large. The size of a small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two. An upper limit of a plurality may be set, for example, by the theoretical limit of oligonucleotides (e.g., capture oligonucleotides) on a surface.

In some aspects, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In aspects, the label is a dye. In aspects, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In aspects, a particular nucleotide type is associated with a particular label, such that identifying the label identifies the nucleotide with which it is associated. In aspects, the label is luciferin that reacts with luciferase to produce a detectable signal in response to one or more bases being incorporated into an elongated complementary strand, such as in pyrosequencing. In aspect, a nucleotide includes a label (such as a dye). In aspects, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In aspects, the detectable moiety is a fluorescent molecule (e.g., acridine dye, cyanine, dye, fluorine dye, oxazine dye, phenanthridine dye, or rhodamine dye). In aspects, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In aspects, the cyanine moiety has 3 methine structures (i.e., cyanine 3 or Cy3). In aspects, the cyanine moiety has 5 methine structures (i.e., cyanine 5 or Cy5). In aspects, the cyanine moiety has 7 methine structures (i.e., cyanine 7 or Cy7).

### Oligonucleotides

An oligonucleotide is a polymer comprised of nucleotides. Oligonucleotides of the disclosure (e.g., an aptamer) may be of any length and include, in various aspects, DNA oligonucleotides, RNA oligonucleotides, analogs thereof, or a combination thereof. In any aspects or aspects described herein, an oligonucleotide is single-stranded, double-stranded, or partially double-stranded.

Nucleotides may include naturally occurring nucleotides and functional analogs thereof. Examples of functional analogs are those that are capable of hybridizing to a nucleic acid in a sequence specific fashion or capable of being used as a template for replication of a particular nucleotide sequence. Naturally occurring nucleotides generally have a backbone containing phosphodiester bonds. An analog structure can have an alternate backbone linkage including any of a variety known in the art. Naturally occurring nucleotides generally have a deoxyribose sugar (e.g., found in DNA) or a ribose sugar (e.g., found in RNA). An analog structure can have an alternate sugar moiety including any of a variety known in the art. Nucleotides can include native or non-native bases. A native DNA can include one or more of adenine, thymine, cytosine and/or guanine, and a native RNA can include one or more of adenine, uracil, cytosine and/or guanine. Any non-native base may be used, such as a locked nucleic acid (LNA) and a bridged nucleic acid (BNA). Example modified nucleotides include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

Oligonucleotides contemplated by the disclosure also include those having at least one modified internucleotide linkage. In some aspects, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. Examples of universal bases include but are not limited to 5′-nitroindole-2′-deoxyriboside, 3-nitropyrrole, inosine and hypoxanthine.

In various aspects, an oligonucleotide of the disclosure, or a modified form thereof, is generally about 5 nucleotides to about 150 nucleotides in length. In further aspects, an oligonucleotide of the disclosure is about 5 to about 125 nucleotides in length, about 5 to about 100 nucleotides in length, about 5 to about 90 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 150 nucleotides in length, about 10 to about 125 nucleotides in length, about 10 to about 100 nucleotides in length, about 10 to about 90 about 10 to about 50 nucleotides in length, about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. Accordingly, in various aspects, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more nucleotides in length. In further aspects, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more nucleotides in length.

As used herein, the term “poly T” or “poly A,” when used in reference to a nucleic acid sequence, is intended to mean a series of two or more thiamine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively. In some aspects, the disclosure contemplates use of a “polyTVN” sequence, which is a poly T sequence followed by a V (any base but a T) and an N. The polyTVN sequence is used, in some aspects, to bias reverse transcription to the base of the poly A tail on the mRNA molecule.

As used herein, the term “immobilized” when used in reference to an oligonucleotide is intended to mean direct or indirect attachment to a surface via covalent or non-covalent bond(s). In certain aspects, covalent attachment can be used, but all that is required is that the oligonucleotides remain stationary or attached to a surface under conditions in which it is intended to use the surface, for example, in applications requiring nucleic acid capture, amplification, and/or sequencing. Oligonucleotides to be used as capture oligonucleotides can be immobilized such that a 3′-end is available for enzymatic extension and at least a portion of the sequence is capable of hybridizing to a complementary sequence. Immobilization can occur via hybridization to a surface attached oligonucleotide, in which case the immobilized oligonucleotide or polynucleotide can be in the 3′-5′ orientation. Alternatively, immobilization of oligonucleotides can comprise use of a selectively cleavable linker. Examples of selectively cleavable linkers include, but are not limited to, biotin-based molecules (e.g., desthiobiotin molecule(s) (ddBio)), PC Linker, and a recognition site for a rare-cutter enzyme. Typically, the selectively cleavable linker can be cleaved by heating, competitive binding, pH change, chemical cleavage, enzymatic cleavage and/or photo-cleavage. Cleaving the selectively cleavable linker results in the release the nucleic acid, or a portion thereof, from the substrate or feature of the substrate.

Certain aspects make use of an inert substrate or matrix (e.g., glass slides, polymer beads etc.) that has been functionalized, for example by application of a layer or coating of an intermediate material comprising reactive groups which permit covalent attachment to biomolecules, such as polynucleotides. Examples of such substrates include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such aspects, the biomolecules (e.g., polynucleotides) may be directly covalently attached to the intermediate material (e.g., the hydrogel) but the intermediate material may itself be non-covalently attached to the substrate or matrix (e.g., the glass substrate). The term “covalent attachment to a substrate” is to be interpreted accordingly as encompassing this type of arrangement.

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “extend,” when used in reference to a nucleic acid, is intended to mean addition of at least one nucleotide or oligonucleotide to the nucleic acid. In particular aspects one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In aspects, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol τ DNA polymerase, Pol κ DNA polymerase, Pol ζ DNA polymerase, Pol γ DNA polymerase, Pol θ DNA polymerase, Pol κ DNA polymerase, or a thermophilic nucleic acid polymerase (e.g. Therminator γ, 9° N polymerase (exo-), Therminator II, Therminator III, or Therminator IX). In aspects, the DNA polymerase is a modified archaeal DNA polymerase. In aspects, the polymerase is a reverse transcriptase. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In aspects, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In aspects, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In aspects, 5′-3′ exonuclease activity refers to the successive removal of nucleotides in double-stranded DNA in a 5′→3′ direction. In aspects, the 5′-3′ exonuclease is lambda exonuclease. For example, lambda exonuclease catalyzes the removal of 5′ mononucleotides from duplex DNA, with a preference for 5′ phosphorylated double-stranded DNA. In other aspects, the 5′-3′ exonuclease is E. coli DNA Polymerase I.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S20₄), or hydrazine (N₂H4)). A chemically cleavable linker is non-enzymatically cleavable. In aspects, the cleavable linker is cleaved by contacting the cleavable linker with a cleaving agent. In aspects, the cleaving agent is a phosphine containing reagent (e.g., TCEP or THPP), sodium dithionite (Na₂S20₄), weak acid, hydrazine (N₂H4), Pd(0), or light-irradiation (e.g., ultraviolet radiation). In aspects, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In aspects, the scissile linkage can be located at any position within the one or more nucleic acid molecules, including at or near a terminal end (e.g., the 3′ end of an oligonucleotide) or in an interior portion of the one or more nucleic acid molecules. In aspects, conditions suitable for separating a scissile linkage include a modulating the pH and/or the temperature. In aspects, a scissile site can include at least one acid-labile linkage. For example, an acid-labile linkage may include a phosphoramidate linkage. In aspects, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In aspects, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In aspects, the scissile site includes at least one uracil nucleobase. In aspects, a uracil nucleobase can be cleaved with a uracil DNA glycosylase (UDG) or Formamidopyrimidine DNA Glycosylase Fpg. In aspects, the scissile linkage site includes a sequence-specific nicking site having a nucleotide sequence that is recognized and nicked by a nicking endonuclease enzyme or a uracil DNA glycosylase. In aspects, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In aspects, when the modified nucleotide is a ribonucleotide, the cleavable site can be cleaved with an endoribonuclease. In aspects, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In aspects, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In aspects, the cleaving agent includes a restriction endonuclease, including, for example a type IIS restriction endonuclease. In aspects, the cleaving agent is an exonuclease (e.g., RecBCD), restriction nuclease, endoribonuclease, exoribonuclease, or RNase (e.g., RNAse I, II, or III). In aspects, the cleaving agent is a restriction enzyme. In aspects, the cleaving agent includes a glycosylase and one or more suitable endonucleases. In aspects, cleavage is performed under alkaline (e.g., pH greater than 8) buffer conditions at between 40° C. to 80° C. (e.g., 65° C.).

### Aptamers

Methods of the present disclosure comprise the use of aptamers, which are oligonucleotides that can specifically bind proteins. In general, the disclosure provides methods in which aptamers specifically bind to a protein in a biological sample. Following the binding of the aptamer to the protein, the aptamer (or portion thereof), or an oligonucleotide that is hybridized to the aptamer, is released from the protein and is captured onto the surface via a capture oligonucleotide. The aptamer sequence may then be copied, amplified, and sequenced to determine (i) identity of the aptamer and therefore the protein and (ii) spatial information of the protein in the biological sample.

As used herein, “aptamer,” and “SOMAmer” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target, and facilitating the reaction between the target and another molecule. In one aspect, the action is specific binding affinity for a target molecule, such target molecule being a three dimensional chemical structure other than a polynucleotide that binds to the nucleic acid ligand through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. Aptamers may be designed with any combination of the base modified nucleotides desired.

As used herein, a “SOMAmer” or Slow Off-Rate Modified Aptamer refers to an aptamer (including an aptamers comprising at least one nucleotide with a hydrophobic modification) with an off-rate (t1/2) of ≥30 minutes. In some aspects, SOMAmers are generated using the improved SELEX methods described in U.S. Pat. No. 7,947,447, entitled “Method for Generating Aptamers with Improved Off-Rates”, which is incorporated herein by reference.

An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

As used herein, the terms “aptamer-target affinity complex”, “aptamer affinity complex” or “aptamer complex” refer to a non-covalent complex that is formed by the interaction of an aptamer with its target molecule. “Aptamer-target affinity complexes”, “aptamer affinity complexes” or “aptamer complexes” refer to more than one such set of complexes. An aptamer-target affinity complex, aptamer affinity complex or aptamer complex can generally be reversed or dissociated by a change in an environmental condition, e.g., an increase in temperature, an increase in salt concentration, or an addition of a denaturant.

In some aspects, a non-covalent complex of an aptamer and its target is provided, wherein the aptamer has a Kd for the target of about 100 nM or less, wherein the rate of dissociation (as given by half-life of the complex; t1/2) of the aptamer from the target is greater than or equal to about 30 minutes; and/or wherein one, several or all pyrimidines in the nucleic acid sequence of the aptamer are modified at the 5-position of the base.

As used herein, “non-specific complex” refers to a non-covalent association between two or more molecules other than an aptamer and its target molecule. Because a non-specific complex is not selected on the basis of an affinity interaction between its constituent molecules, but represents an interaction between classes of molecules, molecules associated in a non-specific complex will exhibit, on average, much lower affinities for each other and will have a correspondingly higher dissociation rate than an aptamer and its target molecule. Non-specific complexes include complexes formed between an aptamer and a non-target molecule, an aptamer and another aptamer, a competitor and a non-target molecule, a competitor and a target molecule, an aptamer and a competitor, and a target molecule and a non-target molecule as well as higher order aggregates of aptamer, target molecule, non-target molecule, surface and competitor.

As used herein, “target molecule,” “analyte,” and “target” are used interchangeably to refer to any molecule of interest to which an aptamer can bind with high affinity and specificity and that may be present in a test sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component that does not substantially alter the identity of the molecule. Exemplary target molecules include proteins, polypeptides, nucleic acids, carbohydrates, lipids, polysaccharides, glycoproteins, hormones, receptors, antigens, antibodies, affibodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. An aptamer may be identified for virtually any chemical or biological molecule of any size, and thus virtually any chemical or biological molecule of any size can be a suitable target. A target can also be modified to enhance the likelihood or strength of an interaction between the target and the aptamer. A target can also be modified to include a tag, as defined above. In exemplary aspects, the target molecule is a protein. See U.S. Pat. No. 6,376,190 entitled “Modified SELEX Processes Without Purified Protein” for methods in which the SELEX target is a peptide.

In various aspects, aptamers of the disclosure comprise one or more elements including but not limited to a protein binding nucleotide sequence, a complementary target nucleotide sequence, an adapter nucleotide sequence or portion thereof, an aptamer barcode nucleotide sequence, a cleavage site, an aptamer-specific nucleotide sequence, or a combination thereof. In some aspects, an aptamer of the disclosure comprises a capture nucleotide sequence and an oligonucleotide that is hybridized to the aptamer comprises an at least partially complementary target nucleotide sequence. As used herein, a “capture nucleotide sequence” is a sequence that is able to hybridize to a target nucleotide sequence on an aptamer or on an oligonucleotide that is hybridized to an aptamer. In some aspects, a capture nucleotide sequence is a homopolymeric nucleotide sequence (e.g., a poly T sequence). As used herein, a “target nucleotide sequence” is a sequence that is able to hybridize to a capture nucleotide sequence (e.g., a capture nucleotide sequence that is present on a capture oligonucleotide). In some aspects, a target nucleotide sequence is a homopolymeric nucleotide sequence (e.g., a poly A sequence).

In some aspects, an aptamer comprises a protein-binding nucleotide sequence, a target nucleotide sequence, and an adapter nucleotide sequence or portion thereof. In further aspects, an aptamer comprises a protein binding nucleotide sequence, a target nucleotide sequence, and an aptamer barcode nucleotide sequence. In yet additional examples, the disclosure provides an aptamer complex comprising (1) an aptamer comprising (i) a capture nucleotide sequence; and (ii) an aptamer-specific nucleotide sequence; and (2) an oligonucleotide hybridized to the aptamer prior to the contacting of the aptamer complex with a protein, the oligonucleotide comprising (i) a target nucleotide sequence; (ii) a sequence complementary to the aptamer-specific nucleotide sequence; and (iii) an aptamer barcode nucleotide sequence.

As described below, aptamers can range from about 20 to about 100 nucleotides in length. In some aspects, the aptamer is about 20, about 30, about 40, about 50, about 60, about 70, about 80, about 90, or about 100 nucleotides in length. In some aspects, the aptamer comprises RNA bases. In some aspects, the aptamer comprises DNA bases. In some aspects, the aptamer comprises RNA and DNA bases.

### Methods

In any of the aspects or aspects of the disclosure, a plurality of aptamers is provided that associate with one or more proteins in a biological sample (e.g., tissue slice), resulting in capture of the aptamers, portion of the aptamers, and/or oligonucleotides hybridized to the aptamers onto the surface. In some aspects, nucleic acids (e.g., mRNA) in a biological sample (e.g., tissue slice) are transferred to and captured onto an array. In any of the aspects or aspects of the disclosure, co-assays providing spatial information regarding proteins and nucleic acids from a biological sample (e.g., tissue slice) are performed.

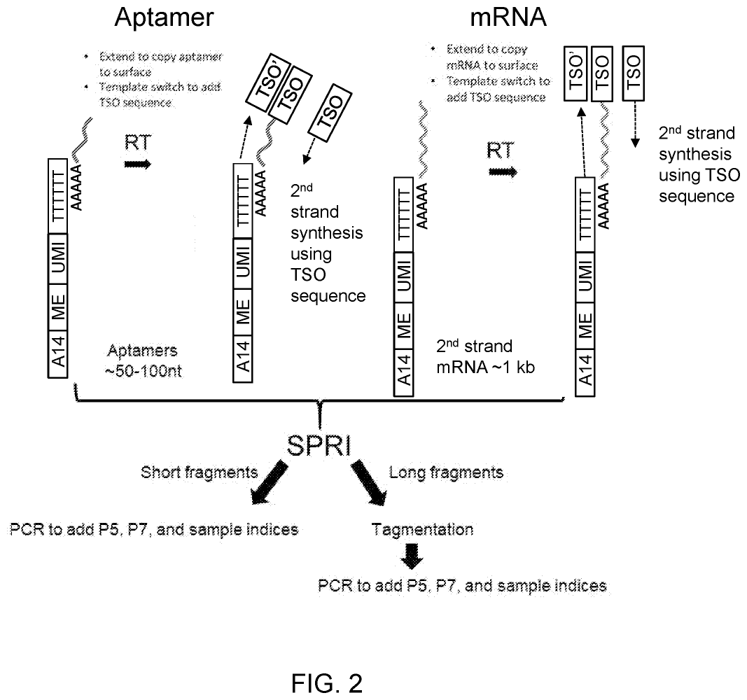

For example, in some aspects a biological sample (e.g., a tissue slice) is placed in contact with a surface and proteins from the biological sample associate with aptamers. In various aspects, the aptamers are then released from the protein and captured onto the surface (e.g., by hybridization to a capture oligonucleotide) and tagged with a spatial address (barcode). In some aspects, the aptamers comprise an aptamer barcode. In some aspects, an oligonucleotide that is hybridized to the aptamer is released following association of the aptamer with a protein and the oligonucleotide is captured onto the surface and tagged with a spatial address (barcode). The spatially-tagged oligonucleotides are released from the array and analyzed, for example, by high throughput next generation sequencing (NGS), such as sequencing-by-synthesis (SBS). In some aspects, a capture oligonucleotide can be a universal capture oligonucleotide hybridizing, e.g., to an adaptor region in a nucleic acid sequencing library, or to the poly-A tail of an mRNA or aptamer. In some aspects, the capture oligonucleotide can be a gene-specific capture oligonucleotide hybridizing, e.g., to a specifically targeted mRNA or cDNA in a sample, such as a TruSeq™ Custom Amplicon (TSCA) oligonucleotide probe (Illumina, Inc.). In some aspects, a surface comprises a plurality of capture oligonucleotides, e.g., a plurality of the same or of different capture oligonucleotides. As described herein, in any of the aspects or aspects of the disclosure, methods described herein provide for co-assay of proteins and nucleic acids (e.g., mRNA) from a biological sample. See, e.g., FIG. 2.

In any of the aspects or aspects of the disclosure, methods as described herein can include a step of extending surface-attached capture oligonucleotides to which aptamers, oligonucleotides, and/or target nucleic acids are hybridized. In aspects where the capture oligonucleotides include barcode sequences, the resulting extended sequences will include the barcode sequences and sequences from the aptamers, oligonucleotides, and/or target nucleic acids (albeit in complementary form). The extended sequences are thus spatially tagged versions of the target nucleic acids from the biological sample.

The sequences of the extended capture oligonucleotides identify what proteins and/or nucleic acids are in the biological sample and where in the biological specimen the proteins and/or nucleic acids are located. It will be understood that other sequence elements that are present in the capture oligonucleotides can also be included in the extended probes. Such elements include, for example, primer binding sites, cleavage sites, other tag sequences (e.g., sample identification tags), capture sequences, or a combination thereof.

Accordingly, in some aspects the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a cleavage site; (c) removing aptamers in the plurality of aptamers that did not associate with a protein in the biological sample; (d) cleaving the plurality of aptamers to release (i) the target nucleotide sequence and (ii) the aptamer barcode nucleotide sequence, thereby resulting in association of the target nucleotide sequence with the capture nucleotide sequence, and thereby preparing the spatial proteome sequencing library. In some aspects, the surface further comprises a blocker nucleic acid that is hybridized to at least a portion of the capture nucleotide sequence. In further aspects, the blocker nucleic acid is removed from the capture oligonucleotide after step (c). In some aspects, the plurality of aptamers is cleaved via ultraviolet radiation, an enzyme, or chemical cleavage. In further aspects, methods of the disclosure further comprise step (e) extending the capture nucleotide sequence to create copies of the individual aptamers, thereby creating extended capture oligonucleotides. In further examples, methods of the disclosure further comprise step (f) adding a template switch oligonucleotide (TSO) to the 3′ end of the extended capture oligonucleotides. By way of example, in some aspects a reverse transcriptase having terminal transferase activity is used, and the reverse transcriptase adds untemplated cytosines to the end of a cDNA molecule. In these aspects, a template switch oligonucleotide (TSO) having complementarity to the added cytosines is included in the reaction, thereby allowing the TSO to hybridize to the end of the cDNA molecule and the reverse transcriptase enzyme uses that as a new template to add bases complementary to the TSO. In some examples, methods of the disclosure further comprise performing a single-strand ligation to append a primer landing site for subsequent second strand synthesis.

Any suitable enzymatic, chemical, or photochemical cleavage reaction may be used to cleave the cleavable site. The cleavage reaction may result in removal of a part or the whole of the strand being cleaved. Suitable cleavage means include, for example, restriction enzyme digestion, in which case the cleavable site is an appropriate restriction site for the enzyme which directs cleavage of one or both strands of a duplex template; RNase digestion or chemical cleavage of a bond between a deoxyribonucleotide and a ribonucleotide, in which case the cleavable site may include one or more ribonucleotides; chemical reduction of a disulfide linkage with a reducing agent (e.g., THPP or TCEP), in which case the cleavable site should include an appropriate disulfide linkage; chemical cleavage of a diol linkage with periodate, in which case the cleavable site should include a diol linkage; generation of an abasic site and subsequent hydrolysis, etc. In aspects, the cleavable site is included in the surface immobilized primer (e.g., within the polynucleotide sequence of the primer). In aspects, one strand of the double-stranded amplification product (or the surface immobilized primer) may include a diol linkage which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In aspects, the diol-cleaving agent is periodate, e.g., aqueous sodium periodate (NaIO4). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine.

A “blocking oligonucleotide,” “blocker oligonucleotide,” “blocking element,” or “blocker nucleic acid” refers to an agent (e.g., polynucleotide, protein, nucleotide) that reduces and/or inhibits hybridization. In aspects, the blocker nucleic acid is a non-extendable oligomer (e.g., a 3′-blocked oligo). A blocking element on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group. In aspects the blocking moiety is not reversible (e.g., the blocking element including a blocking moiety irreversibly prevents extension). In aspects, the blocker nucleic acid includes an oligo having a 3′ dideoxynucleotide or similar modification to prevent extension by a polymerase and is used in conjunction with a non-strand displacing polymerase. In another example implementation, the blocking element includes one or more modified nucleotides including a cleavable linker (e.g., linked to the 5′, 3′, or the nucleobase) containing PEG, thereby blocking the extension. In another example implementation, the blocker nucleic acid includes one or more modified nucleotides linked to biotin, to which a protein (e.g., streptavidin) can be bound, thereby blocking polymerase extension. In another example implementation, the blocker nucleic acid includes a modified nucleotide, such as iso dGTP or iso dCTP, which are complementary to each other. In a reaction of polymerization lacking the appropriate complementary modified nucleotides, the extension of a primer is halted. In another example implementation, the blocker nucleic acid includes one or more sequences which is recognized and bound by one or more single-stranded DNA-binding proteins, thereby blocking polymerase extension at the bound site. In another example implementation, the blocker nucleic acid includes one or more sequences which are recognized and bound by one or more short RNA or PNA oligonucleotides, thereby blocking the extension by a DNA polymerase that cannot strand displace RNA or PNA.

In further aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a truncated adapter nucleotide sequence; (c) removing aptamers in the plurality of aptamers that did not associate with a protein in the biological sample; (d) eluting the individual aptamers from the individual proteins, thereby resulting in association of the target nucleotide sequence with the capture nucleotide sequence, and thereby preparing the spatial proteome sequencing library. In some aspects, the surface further comprises a blocker nucleic acid that is hybridized to at least a portion of the capture nucleotide sequence. In further aspects, the blocker nucleic acid is removed from the capture oligonucleotide after step (c). In some aspects, methods of the disclosure further comprise step (e) extending the capture nucleotide sequence to create copies of the individual aptamers, thereby creating extended capture oligonucleotides. In further examples, methods of the disclosure further comprise step (f) hybridizing the truncated adapter nucleotide sequence to a full length adapter nucleotide sequence primer and extending to synthesize a second strand.

In further aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamer complexes in the plurality of aptamer complexes with individual proteins in the biological sample, wherein each aptamer complex in the plurality of aptamer complexes comprises: (1) an aptamer comprising (i) the capture nucleotide sequence; and (ii) an aptamer-specific nucleotide sequence; and (2) an oligonucleotide hybridized to the aptamer prior to the contacting, the oligonucleotide comprising (i) the target nucleotide sequence; (ii) a sequence complementary to the aptamer-specific nucleotide sequence; and (iii) an aptamer barcode nucleotide sequence, wherein after the association of individual aptamer complexes in the plurality of aptamer complexes with individual proteins in the biological sample, the oligonucleotide is released from the aptamer thereby resulting in association of the target nucleotide sequence of the released oligonucleotide with the capture nucleotide sequence of a capture oligonucleotide of the plurality of capture oligonucleotides; thereby preparing the spatial proteome sequencing library. In some aspects, the aptamer-specific nucleotide sequence is about 5 to about 20 nucleotides in length. In further aspects, the aptamer-specific nucleotide sequence is about 10 nucleotides in length. In some aspects, the association of individual aptamer complexes in the plurality of aptamer complexes with individual proteins in the biological sample results in release of the oligonucleotide from the aptamer. In further aspects, after the association of individual aptamer complexes in the plurality of aptamer complexes with individual proteins in the biological sample, a condition is changed thereby resulting in release of the oligonucleotide from the aptamer. In various examples, the condition is temperature, pH, or salt concentration. In some aspects, formamide is added thereby resulting in release of the oligonucleotide from the aptamer.

In any of the aspects or aspects of the disclosure, the surface further comprises a blocker nucleic acid that is hybridized to at least a portion of the capture nucleotide sequence. In some aspects, the blocker nucleic acid is removed from the capture oligonucleotide after the contacting. In some examples, the blocker oligonucleotide is removed from the capture oligonucleotide by exonuclease digestion. In various aspects, the exonuclease digestion is performed using T7 exonuclease or lambda exonuclease.

In further aspects, the disclosure provides a method of preparing a spatial proteome sequencing library from a biological sample, the method comprising: (a) providing a surface comprising: a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence; (b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; and (ii) an aptamer barcode nucleotide sequence; (c) removing aptamers in the plurality of aptamers that did not associate with a protein in the biological sample; (d) eluting the individual aptamers from the individual proteins, thereby resulting in hybridization of the target nucleotide sequence with the capture nucleotide sequence, and thereby preparing the spatial proteome sequencing library. In some aspects, the plurality of capture oligonucleotides comprises a cleavable site at the 5′ end. In some aspects, step (d) comprises competitive elution with aptamers or digesting the proteins in the biological sample. In some aspects, step (d) further comprises contacting at least one aptamer of the plurality of aptamers with a blocker nucleic acid, thereby forming a blocked aptamer, wherein the blocker nucleic acid is complementary to the target nucleotide sequence, and wherein the blocked aptamer is unable to associate with the capture nucleotide sequence. In some aspects, the surface further comprises a blocker nucleic acid that is hybridized to at least a portion of the capture nucleotide sequence. In some aspects, the blocker nucleic acid is removed from the capture oligonucleotide after step (c). In some aspects, the method further comprises (e) extending the capture nucleotide sequence to create copies of the individual aptamers, thereby creating extended capture oligonucleotides. In some aspects, step (e) further comprises hybridizing a plurality of aptamer barcoded oligonucleotides to the extended capture oligonucleotides, and extending the extended capture oligonucleotides, thereby creating a plurality of barcoded capture oligonucleotides, wherein each of the aptamer barcoded oligonucleotides comprises at least a portion of an individual aptamer sequence. In some aspects, the plurality of aptamer barcoded oligonucleotides comprise a plurality of aptamer blocker nucleic acids, wherein each of the aptamer blocker nucleic acids comprises at least a portion of an individual aptamer sequence.

In some aspects, between 10% to 90% of a plurality of aptamers specific for a protein (e.g., an abundant protein) are hybridized to an aptamer blocker nucleic acid. In some aspects, about 10%, 20%, 30%, 40%, 50%, 60%, 70%, 80%, 90%, or greater than 90% of the plurality of aptamers specific for a protein are hybridized to an aptamer blocker nucleic acid. It will be understood that the amount of aptamer blocker nucleic acid included in the method is adjustable based upon the known abundance of a given target protein.

In some aspects, the method further comprises contacting a second plurality of aptamers to the biological sample on the surface, the contacting resulting in association of individual aptamers in the second plurality of aptamers with individual proteins in the biological sample, wherein each aptamer in the second plurality of aptamers comprises a detectable moiety. In some aspects, the detectable moiety is a fluorophore. In some aspects, each aptamer in the second plurality of aptamers comprises the target nucleotide sequence and a truncated adapter nucleotide sequence. In some aspects, each aptamer in the second plurality of aptamers further comprises an aptamer barcode nucleotide sequence. In some aspects, after contacting the second plurality of aptamers to the biological sample on the surface, the method further comprises imaging the biological sample, thereby obtaining an image of the biological sample. In some aspects, the method does not comprise contacting the biological sample with hematoxylin and eosin (H&E) staining reagents. In some aspects, at least one aptamer in the second plurality of aptamers is specific for a cell membrane-associated protein. In some aspects, at least one aptamer in the second plurality of aptamers is specific for a nuclear membrane-associated protein. In some aspects, at least one aptamer in the second plurality of aptamers is specific for a cell membrane-associated protein and at least one aptamer in the second plurality of aptamers is specific for a nuclear membrane-associated protein, and wherein the at least one aptamer specific for the nuclear membrane-associated protein comprises a different detectable moiety than the at least one aptamer specific for the cell membrane-associated protein. In some aspects, the at least one aptamer specific for the nuclear membrane-associated protein comprises a different aptamer barcode nucleotide sequence than the at least one aptamer specific for the cell membrane-associated protein.

In some aspects, the cell membrane-associated protein is E-cadherin, N-cadherin, or a Na⁺/K⁺-ATPase. Additional examples of cell membrane-associated proteins that may be targeted by the aptamers of the present disclosure include, but are not limited to, G-protein-coupled receptors, epidermal growth factor receptors, P-cadherin, and R-cadherin. In some aspects, the nuclear membrane-associated protein is a nuclear pore complex protein. Examples of nuclear pore complex proteins and nuclear membrane-associated proteins that may be targeted by the aptamers of the present disclosure include, but are not limited to, Nup62, Nup107-160, Nup155, karyopherins, and Tpr.

In any of the aspects or aspects of the disclosure, the biological sample is from a mammal. In various examples, the biological sample is from a human. In some aspects, the biological sample is from a mouse.

In some aspects, the disclosure also provides a method of identifying a disorder in a subject having or at risk of having the disorder comprising: i) generating a spatial proteomic and/or transcriptomic (e.g., mRNA) library from a biological sample from the subject according to the methods of the disclosure, ii) comparing proteomic and/or transcriptomic information from the sample proteomic and/or transcriptomic library to a control proteomic and/or transcriptomic library, iii) identifying a genetic variation in the sample proteomic and/or transcriptomic library associated with the disease. In some aspects, the disorder is a neurodegenerative disorder, a genetic defect, or cancer. In some aspects, the disorder is Alzheimer's disease.

In any of the aspects or aspects of the disclosure, a method of preparing a spatial proteomic and/or transcriptomic library as described herein further comprises polymerase chain reaction (PCR) amplification of aptamers or other oligonucleotides that are captured on a surface. In further aspects, a TSO sequence is used as a primer landing site for second strand synthesis. This product is dehybridized from the surface and PCR is performed on this product. Alternatively, the extended surface oligonucleotides can be cleaved and PCR can be performed on this product. Sequencing adapters may be added on to this product, and then they are put on a second flow cell for standard sequencing (e.g., Illumina sequencing). In any of the aspects or aspects of the disclosure, PCR amplification is followed by sequencing of the amplified products to determine the identity and/or location of the protein and/or mRNA in the biological sample.

Thus, methods described herein can be used in conjunction with a variety of nucleic acid sequencing techniques. In aspects, the sequencing includes sequencing-by-synthesis, sequencing-by-binding, sequencing by ligation, or pyrosequencing. In any of the aspects or aspects of the disclosure, and as described above, sequencing may occur on a second flow cell rather than the initial surface (e.g., flow cell) onto which a biological sample is placed. Particularly applicable techniques are those wherein nucleic acids are attached at fixed locations on a surface (e.g., in an array) such that their relative positions do not change and wherein the array is repeatedly imaged. Aspects in which images are obtained in different color channels, for example, coinciding with different labels used to distinguish one nucleotide base type from another are particularly applicable. In some aspects, the process to determine the nucleotide sequence of a target nucleic acid can be an automated process. Preferred aspects include sequencing-by-synthesis (“SBS”) techniques. “Sequencing-by-synthesis (“SBS”) techniques” generally involve the enzymatic extension of a nascent nucleic acid strand through the iterative addition of nucleotides against a template strand. In traditional methods of SBS, a single nucleotide monomer can be provided to a target nucleotide in the presence of a polymerase in each delivery. However, in the methods described herein, more than one type of nucleotide monomer can be provided to a target nucleic acid in the presence of a polymerase in a delivery.

To further illustrate the present disclosure, examples are given herein. It is to be understood that these examples are provided for illustrative purposes and are not to be construed as limiting the scope of the present disclosure.

### EXAMPLES

### Example 1

### Methods for Spatial Protein/mRNA Co-Assays

Methods of assaying the proteomic and mRNA content of biological samples are provided herein. The methods include those in which the proteomic and mRNA content of a biological sample are co-assayed. See, e.g., FIG. 2.

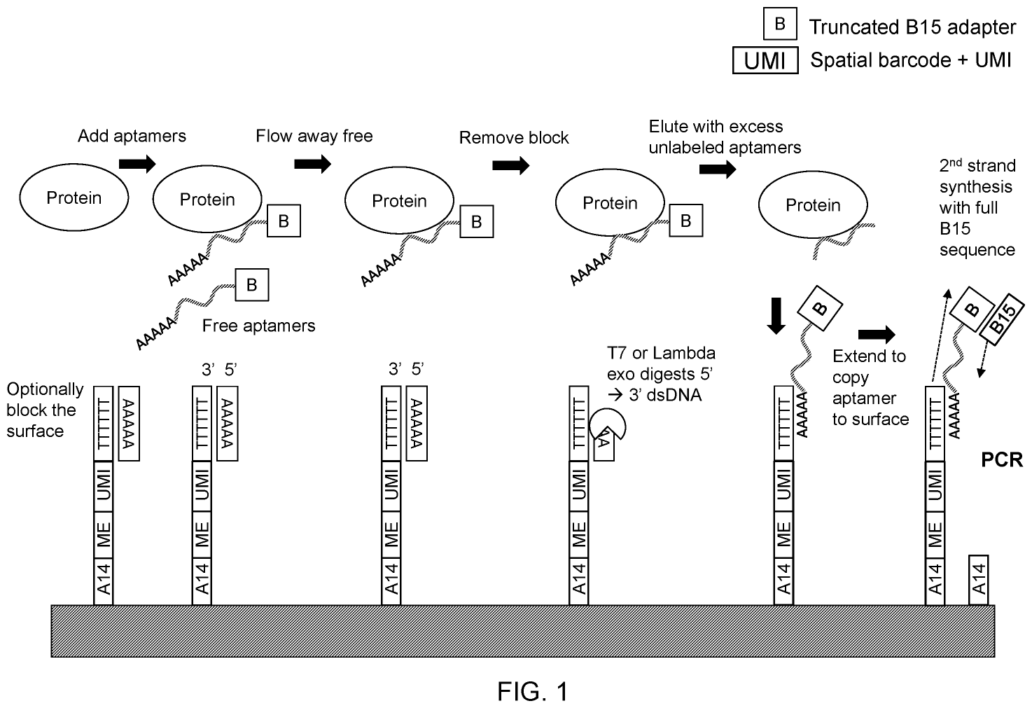

A schematic workflow of one of the methods is shown in FIG. 1. FIG. 1 shows a spatial proteomics workflow using aptamers tagged with a polyA sequence at the 3′ end and a truncated B15 adapter at the 5′ end. A truncated adapter may be used to minimize the extra bases that are added to the aptamers, as these extra bases may impact aptamer-protein binding. The surface capture oligonucleotides have A14 and ME sequences for downstream library preparation steps, as well as a spatial barcode, a UMI, and an oligo-dT sequence to enable capture of polyA-mRNA as well as the tagged aptamers.

At the start of the protocol, the oligo-dT sequence is optionally blocked using a polyA oligonucleotide. Aptamers are added to the tissue section and bind to their target proteins. Free aptamers cannot bind to the surface oligonucleotides because of the polyA block. After washing away the free aptamers, the block is removed using a 5′ to 3′ exonuclease such as T7 or Lambda Exo. The tagged aptamers are then eluted from their targets by addition of excess unlabeled aptamer, and the tagged aptamers then diffuse to the surface and are captured by the surface capture oligonucleotides. An enzyme (e.g., reverse transcriptase or a polymerase) can then be used to copy the aptamer sequence onto the surface capture oligonucleotide. In some aspects, a RNA aptamer is used and the enzyme is a reverse transcriptase. In some aspects, a DNA aptamer is used and the enzyme is a polymerase. In further aspects, a DNA aptamer/mRNA co-assay is performed and the enzyme is a reverse transcriptase. The complementary strand can then be generated using the truncated B15 sequence as a primer, and this complementary strand can be dehybridized from the surface. Alternatively, the capture oligonucleotide/aptamer molecule can be cleaved from the surface. PCR is then performed to add P5/P7 sequences and sample indices to generate the final library. The aptamer sequence (which is unique for every protein target) can then be used to identify the physical location of the target protein using the spatial barcode sequence.

Another workflow method is depicted in FIG. 2. FIG. 2 shows how a workflow of the disclosure can be modified to be compatible with a protein/mRNA co-assay. Here, a template switching reverse transcriptase (RT) is used to add a template switch oligonucleotide (TSO) sequence to the 3′ end of the cDNA or the copied aptamer sequence. This TSO sequence then acts as a primer landing site for copying the extended capture oligonucleotide. This second strand is dehybridized from the surface, and the shorter aptamer sequences are separated from the longer cDNA sequences using a solid-phase reversible immobilization (SPRI) step. The short fragments then undergo PCR to add the rest of the sequencing adapters, whereas the long fragments are tagmented, followed by PCR to add P5/P7 and sample indices. In some aspects, a ligation-based preparation is used.

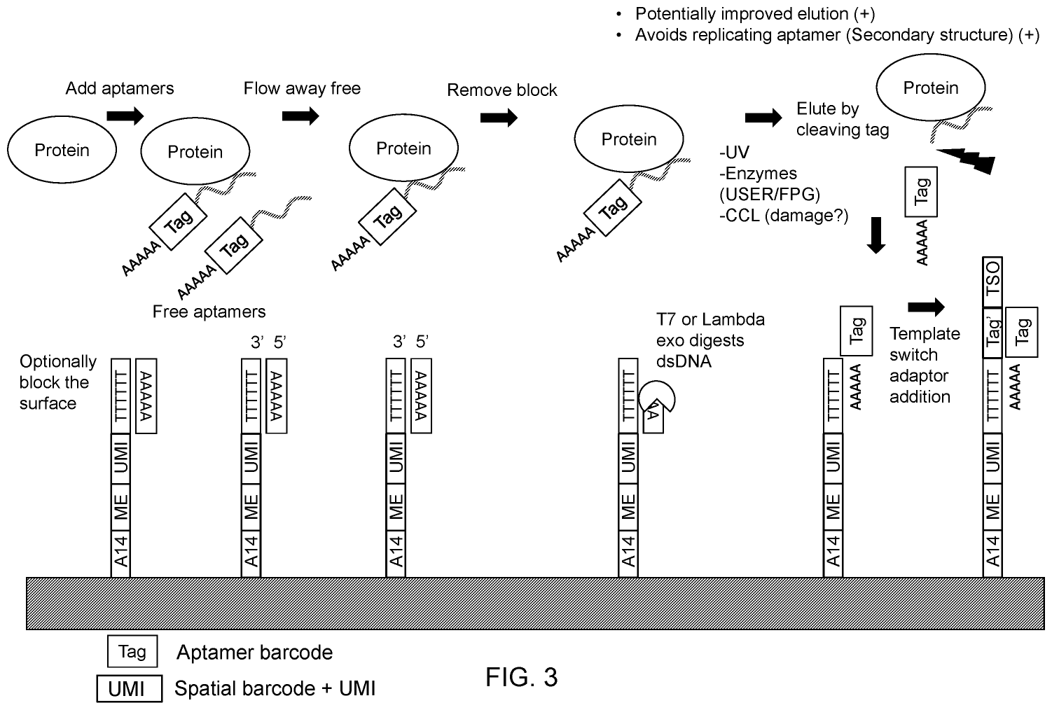

A further workflow method is presented in FIG. 3. FIG. 3 shows a variation of the methods provided herein using aptamers that contain cleavable tags. Here the aptamers contain a cleavage site, followed by a tag sequence that is unique to the protein target, and a polyA tail. In this method, after binding the aptamers to the target proteins, the tag is cleaved from the aptamers and is captured on the surface oligonucleotides. Cleavage can be accomplished using a photocleavable linker in the oligonucleotide activated by, for example, UV light, through enzymatic cleavage by USER or FPG or other targeted endonucleases, or through chemical cleavage. The aptamer is then copied to the surface capture oligonucleotide. This method may lead to improved elution of the aptamer and avoids replicating the aptamer (which may be difficult as aptamers have stable secondary structure).

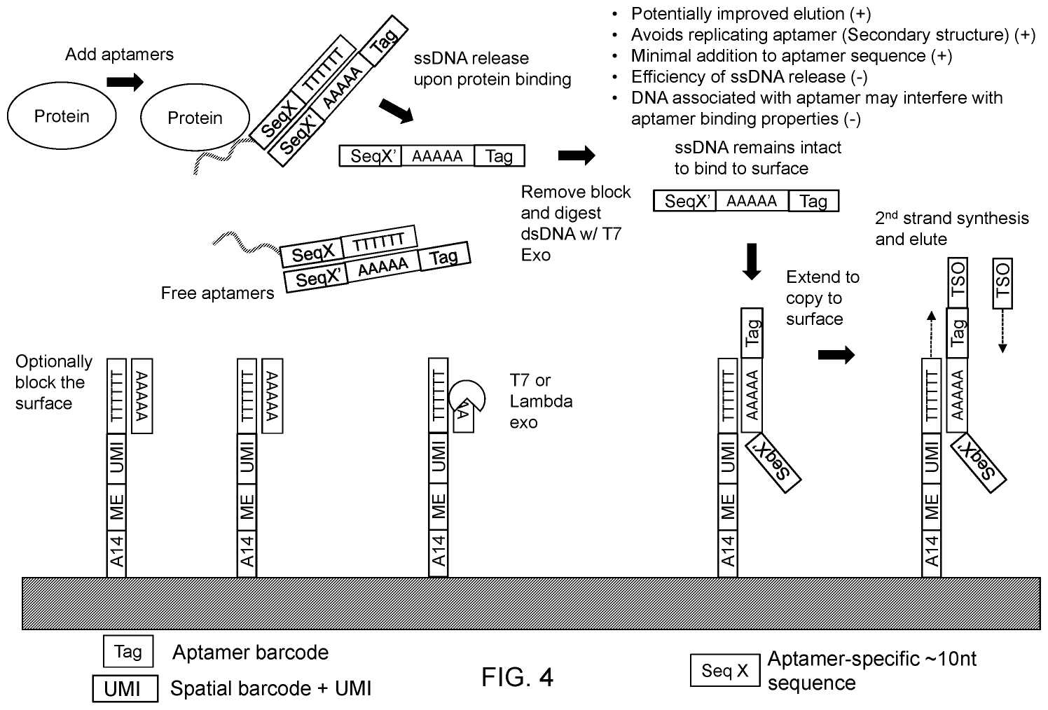

An additional workflow method is shown in FIG. 4. FIG. 4 shows another variation of the methods provided herein using aptamers bound to single-stranded DNA. Aptamers undergo a conformational change when binding their target proteins, and previous work has shown that a single-stranded DNA reporter bound to the aptamer can be released upon binding of the aptamer to its target protein. In this approach, single-stranded at least partially complementary DNA (ssDNA) molecules are bound to the aptamers to form aptamer complexes, and the aptamer complexes are added to a surface before the biological sample (e.g., tissue section) is placed on the surface. In some aspects, single-stranded at least partially complementary DNA (ssDNA) molecules are bound to the aptamers to form aptamer complexes, and the aptamer complexes are added to a surface after a biological sample (e.g., tissue section) is added to the surface. Binding of the aptamers to their target proteins leads to release of the complementary ssDNA molecule, which is then captured on the surface by the capture oligonucleotide. This approach may be beneficial as it minimizes the length of sequence that needs to be added to the aptamer, and doesn't require specific aptamer elution conditions as binding results in elution of the ssDNA molecule.

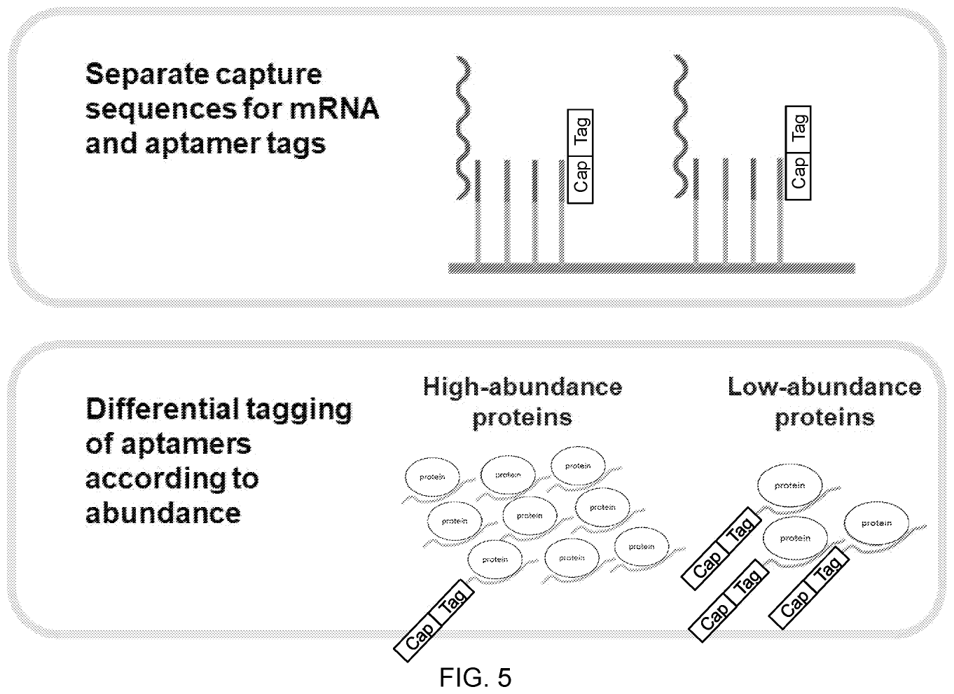

Another workflow is depicted in FIG. 5. FIG. 5 addresses the problem of the large dynamic range and copy number of proteins. Because protein levels are so much higher than mRNA levels, and span a broader dynamic range, it would be useful to have a mechanism to ensure that mRNA transcripts can be captured along with aptamers. To overcome this issue, the present disclosure contemplates use of separate capture oligonucleotide sequences for mRNA and aptamer tags (FIG. 5, top image). To address the large dynamic range of protein expression, the present disclosure contemplates use of a mixture of tagged and untagged aptamers at known ratios for highly abundant proteins, so that only a small percentage of these aptamers could be captured on the surface (FIG. 5, bottom image). Only tagged aptamers are used for low-abundance proteins.

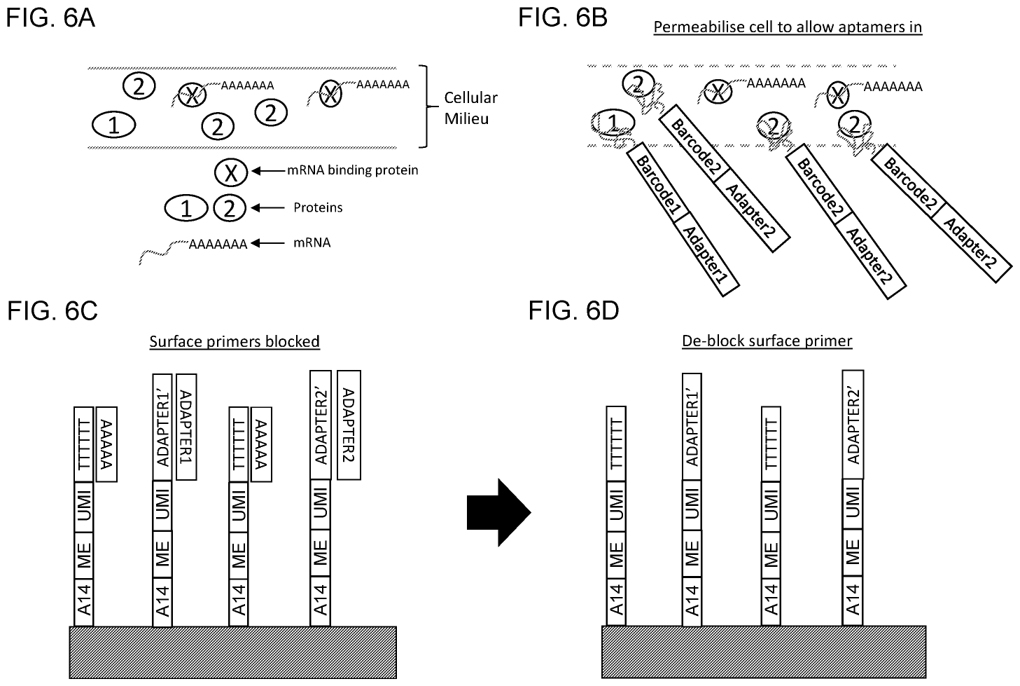

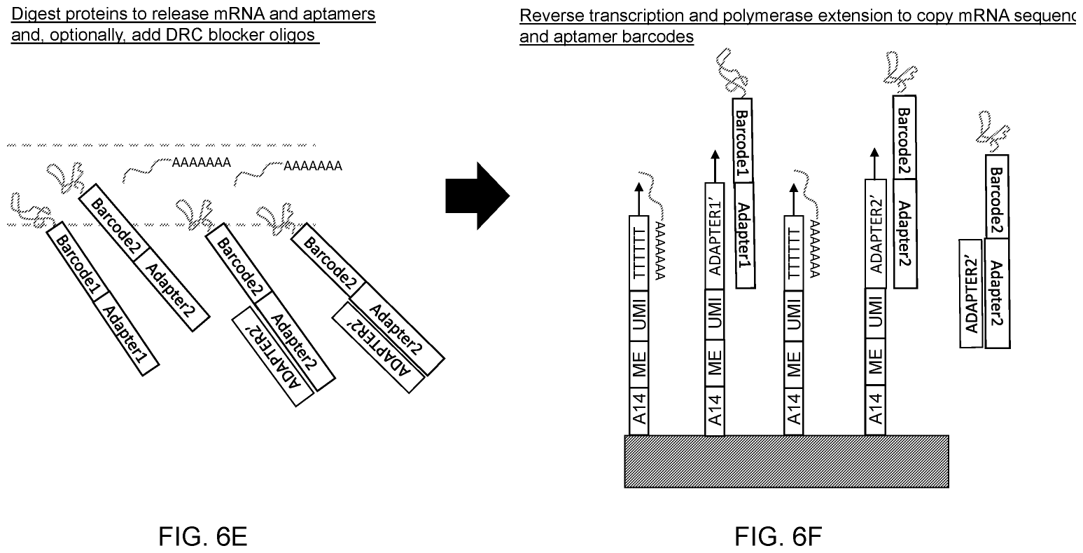

An additional workflow is depicted in FIGS. 6A-6F. FIGS. 6A-6F show an exemplary variation of a workflow of the disclosure using modified aptamers including a unique barcode and an adapter sequence, wherein blocker oligonucleotides complementary for the aptamer adapter sequence are included to accommodate a large dynamic range of protein abundance (also referred to herein as dynamic range control). FIG. 6A is a representative illustration of the cellular milieu including a mixure of proteins (labeled as “1”, “2”, and “X” for mRNA binding protein) and mRNA (depicted as a single-stranded oligonucleotide with a poly-A tail). FIG. 6B shows a step of permeabilizing the cell (e.g., the cell or cells of a tissue section) to allow modified aptamers into the cells, wherein the aptamers are targeting protein 1 and protein 2. The aptamers targeting each protein may include, from 5′ to 3′, a protein-specific aptamer sequence, a barcode sequence (e.g., a barcode1 sequence or a barcode2 sequence), and an adapter sequence (e.g., an adapter1 sequence or an adapter2 sequence). A wash step (not show) removes unbound aptamers, for example, a wash with a non-ionic detergent. Examples of non-ionic detergents include, but are not limited to, Triton™ X-100, Tween®20, Brij® 35, and Brij® 58.

FIG. 6C shows immobilized surface primers hybridized to blocking oligonucleotides. The surface capture oligonucleotides have A14 and ME sequences for downstream library preparation steps, as well as a spatial barcode, a UMI, and an oligo-dT sequence to enable capture of polyA-mRNA as well as the tagged aptamers. FIG. 6D shows a step of deblocking the surface primers to expose a free 3′ end. For example, the block is removed using a 5′ to 3′ exonuclease such as T7 or Lambda Exo, or by exposing the surface of the solid support to denaturing conditions (e.g., heat and/or a chemical denaturant such as formamide) to dehybridize the blocker oligo.

FIG. 6E shows a step of digesting the proteins to release the mRNA and aptamers, and, optionally, adding dynamic range compression (DRC) blocker oligonucleotides specific for abundant protein aptamers. Proteins may be digested, for example, by a proteinase such as proteinase K. Additional enzymes such as a lipase may help facilitate retrieval of the aptamers from the permeabilized tissue/cells. DRC blocker oligonucleotides, as shown in FIG. 6E, may be complementary to one or more adapter sequences of the modified aptamers, forming a blocker-aptamer complex that blocks downstream hybridization of the aptamer with an immobilized surface primer. In some aspects, between 10% to 90% of a plurality of aptamers specific for a protein (e.g., an abundant protein) are hybridized to a blocker oligonucleotide. In some aspects, about 10%, 20%, 30%, 40%, 50%, 60%, 70%, 80%, 90%, or greater than 90% of the plurality of aptamers specific for a protein are hybridized to a blocker oligonucleotide. In some aspects, the blocker oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, or combinations thereof.

In some aspects, the blocker nucleic acid (i.e., the DRC blocker oligo) includes one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleic acids. In aspects, the blocker nucleic acid includes 10 to 15 locked nucleic acids (LNAs). In aspects, the blocker nucleic acid includes one or more phosphorothioates at the 5′ end. In aspects, the blocker nucleic acid includes one or more LNAs at the 5′ end. In aspects, the blocker nucleic acid includes two or more consecutive LNAs at the 3′ end. In aspects, the blocker nucleic acid includes two or more consecutive LNAs at the 5′ end. In aspects, the blocker nucleic acid includes a plurality (e.g., 2 to 10) of synthetic nucleotides (e.g., LNAs) and a plurality (e.g., 2 to 10) canonical or native nucleotides (e.g., dNTPs).

FIG. 6F shows a step of capturing the released mRNA and aptamer barcodes on the immobilized surface primers followed by RT-PCR/PCR to generate covalently attached complements of the mRNA and aptamer barcodes. Aptamers hybridized to DRC blocker oligonucleotides are not captured by the surface primers, and may be washed away. PCR is then performed to add P5/P7 sequences and sample indices to generate the final library. The aptamer sequence (which is unique for every protein target) can then be used to identify the physical location of the target protein using the spatial barcode sequence. Barcodes present in the mRNA capture primers or differences in the sequences themselves allow mRNA sequences to be distinguished from the aptamer sequences.

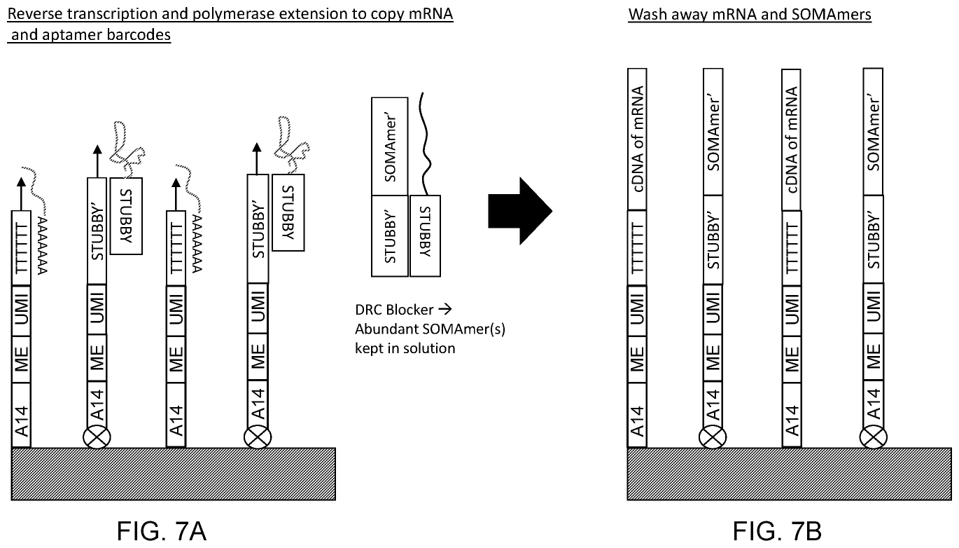

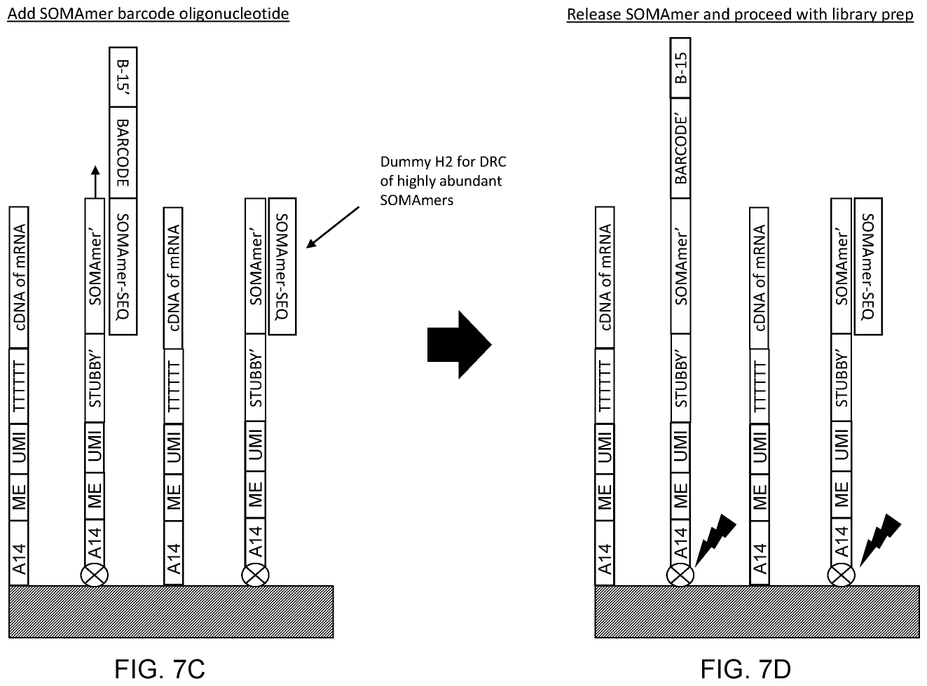

An alternate workflow is depicted in FIGS. 7A-7D. FIGS. 7A-7D show an exemplary variation of a workflow of the disclosure using DRC blockers to maintain abundant aptamers (e.g., SOMAmers®; SomaLogic) in solution, rather than being captured on the surface of the solid support. Tissue sections are permeabilized to allow access for aptamers to enter the cells without losing mRNA. Following the addition of aptamers, unbound aptamers are wash away, for example, using a non-ionic detergent. Examples of non-ionic detergents include, but are not limited to, Triton™ X-100, Tween®20, Brij® 35, and Brij® 58. Cellular proteins are then digested, e.g., with proteinase K, releasing the protein-bound aptamers and any protein-bound mRNA. Immobilized capture oligonucleotides on the solid support are then unblocked, exposing a free 3′ end, allowing hybridization to the mRNA and aptamer sequences.

As shown in FIG. 7A, following cellular permeabilization, aptamer binding, and protein digestion, released mRNA and SOMAmers are captured and copied onto the immobilized surface primers. STUBBY corresponds to a universal adapter sequence at the 3′ end of the aptamer (i.e., the SOMAmer). This is followed by addition of a DRC blocker to the SOMAmer (i.e., a blocker oligo including a complementary sequence to the STUBBY sequence and a complementary sequence to the SOMAmer sequence). FIG. 7B shows a step of, following replication, washing away unbound mRNA and SOMAmers, leaving behind immobilized, extended surface primers including cDNA of the mRNA or complements of the STUBBY and SOMAmer sequences. FIG. 7C shows a step of adding a SOMAmer barcode to the extended surface primers including the complement of the SOMAmer sequence, wherein am oligo including a SOMAmer-SEQ sequence, barcode sequence, and B-15′ adapter sequence is hybridized to the extended surface primer and copied. This oligo may include, from 5′ to 3′, a B-15′ adapter sequence, a barcode sequence, and a sequence complementary to the SOMAmer sequence (e.g., SOMAMER-SEQ). A “dummy” oligo may also be included, which lacks a barcode sequence and B-15′ sequence, and may act as a DRC blocker for preventing replication and sequencing of highly abundant SOMAmers.

FIG. 7D shows a cleavage step to release the extended surface primers including the SOMAmer barcode and B-15 adapter sequence, followed by library prep steps. Cleavage can be accomplished using a photocleavable linker in the oligonucleotide activated by, for example, UV light, through enzymatic cleavage by USER or FPG or other targeted endonucleases, or through chemical cleavage.

### Example 2

### Methods for Spatial Targeting of Cellular Membranes

Methods of using fluorescently-labeled aptamers for assaying a biological sample, for example, cellular and nuclear membranes, are provided herein. The methods include those in which the proteomic and mRNA content of a biological sample are co-assayed.

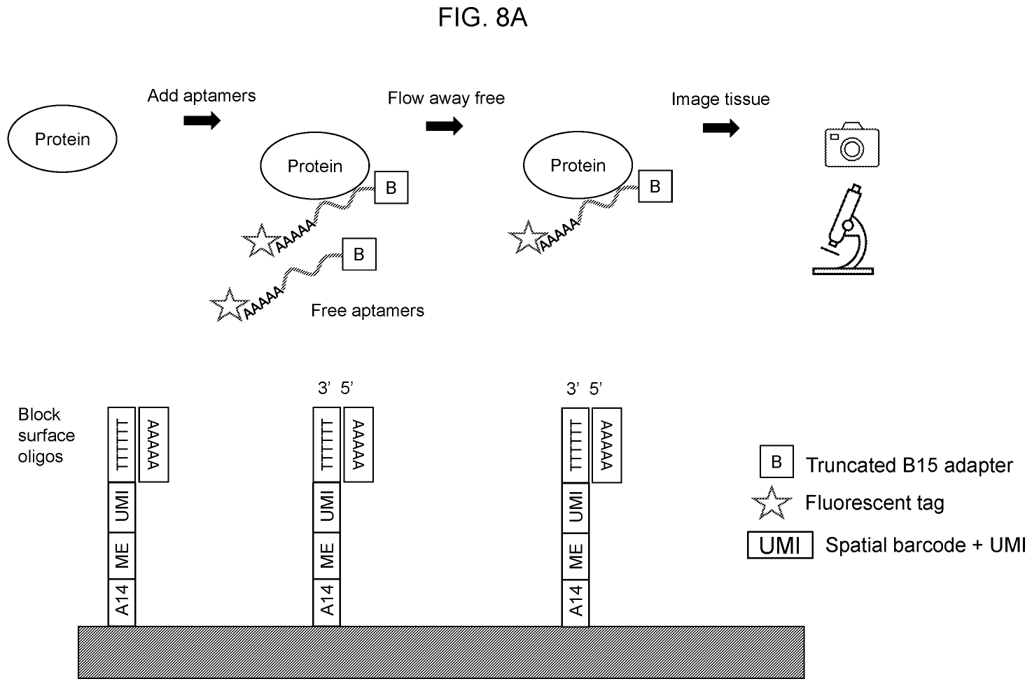

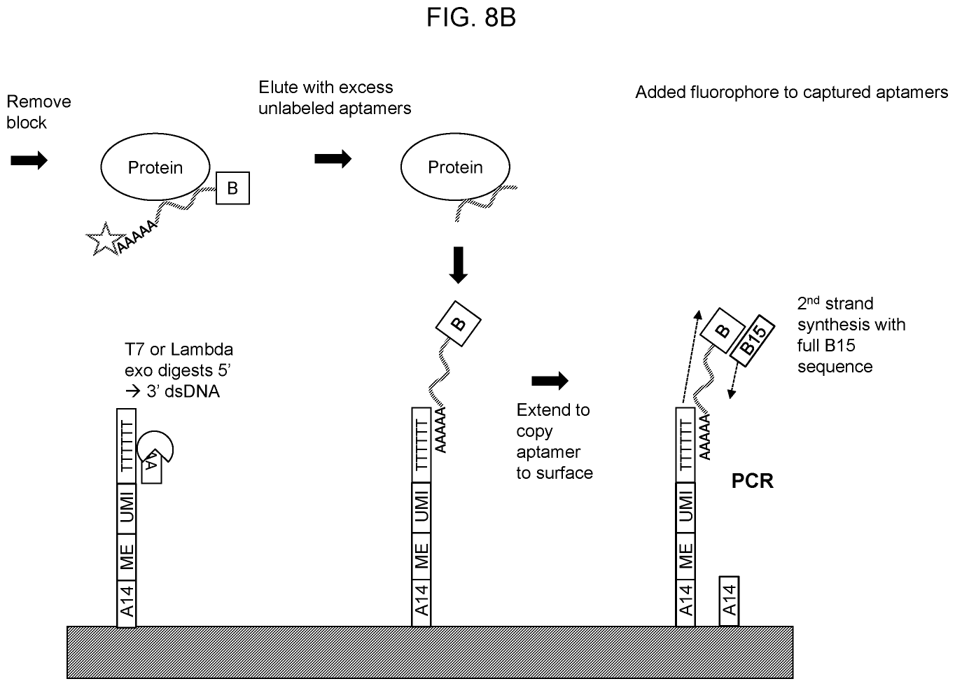

A schematic workflow of one of the methods is shown in FIGS. 8A-8B. FIGS. 8A-8B show an exemplary variation of workflow of the disclosure using fluorescently-labeled aptamers targeting cell membranes, for examples, for cell segmentation analysis and image alignment processing. FIG. 8A shows the steps of adding the fluorescently-labeled aptamers to proteins, flowing away any unbound aptamers, and then imaging the tissue to visualize the bound aptamers. A fluorescent microscope, for example, may be used to image the bound fluorescently-labeled aptamers. The labeled aptamers include a poly-A sequence at the 3′ end (e.g., next to the fluorophore), and a truncated B15 adapter at the 5′ end. A truncated adapter may be used to minimize the extra bases that are added to the aptamers, as these extra bases may impact aptamer-protein binding. The surface capture oligonucleotides have A14 and ME sequences for downstream library preparation steps, as well as a spatial barcode, a UMI, and an oligo-dT sequence to enable capture of polyA-mRNA as well as the tagged aptamers.

The immobilized surface primers on the solid support are blocked with, for example, complementary blocking oligonucleotides. At the start of the protocol, the oligo-dT sequence is optionally blocked using a polyA oligonucleotide. Aptamers are added to the tissue section and bind to their target proteins. Free aptamers cannot bind to the surface oligonucleotides because of the poly A block. FIG. 8B shows the step of removing the blocking oligo from the surface primers with, for example, T7 or lambda exonuclease. The tagged aptamers are then eluted from their targets by addition of excess unlabeled aptamer, and the tagged aptamers then diffuse to the surface and are captured by the surface capture oligonucleotides. An enzyme (e.g., reverse transcriptase or a polymerase) can then be used to copy the aptamer sequence onto the surface capture oligonucleotide. In some aspects, a RNA aptamer is used and the enzyme is a reverse transcriptase. In some aspects, a DNA aptamer is used and the enzyme is a polymerase. In further aspects, a DNA aptamer/mRNA co-assay is performed and the enzyme is a reverse transcriptase. The complementary strand can then be generated using the truncated B15 sequence as a primer, and this complementary strand can be dehybridized from the surface. Alternatively, the capture oligonucleotide/aptamer molecule can be cleaved from the surface. PCR is then performed to add P5/P7 sequences and sample indices to generate the final library. The aptamer sequence (which is unique for every protein target) can then be used to identify the physical location of the target protein using the spatial barcode sequence.

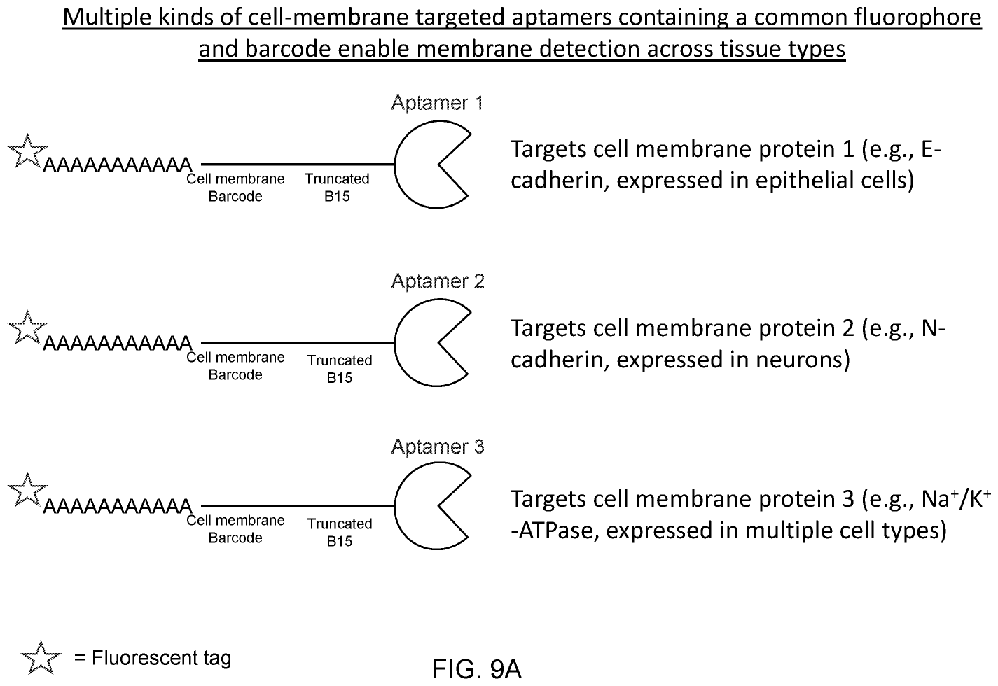

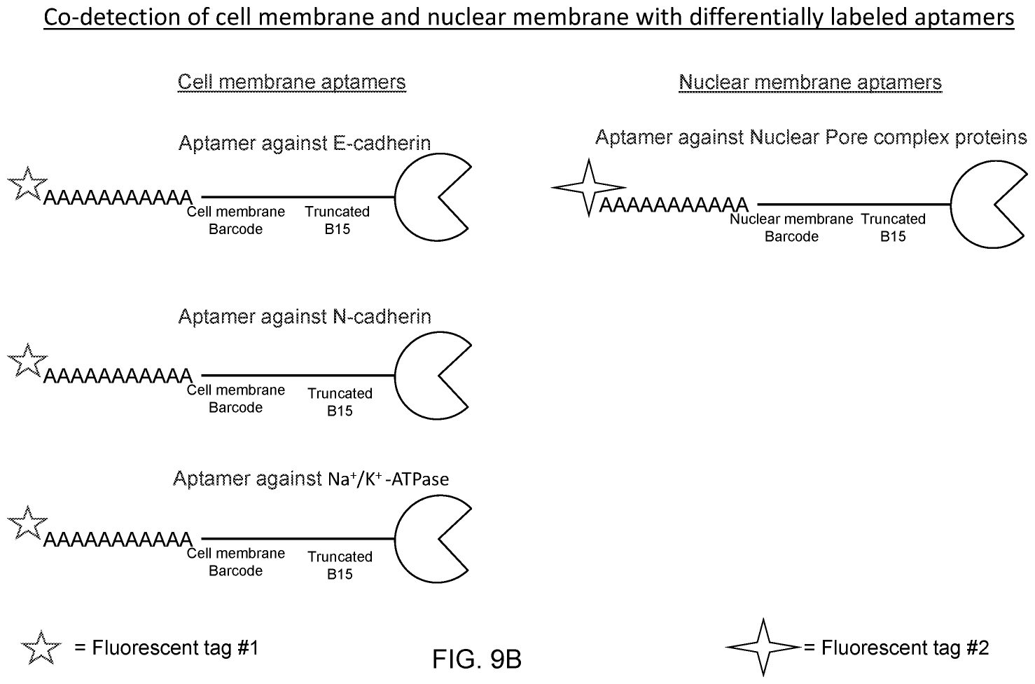

FIGS. 9A-9B show various examples of cell-membrane targeted aptamers and nuclear-membrane targeting aptamers. FIG. 9A shows multiple types of cell-membrane targeted aptamers containing a common fluorophore and barcode to, for example, enable membrane detections across different tissue types. Each aptamer may contain a cell membrane barcode and a truncated B15 adapter sequence. FIG. 9B shows exemplary aptamers for the co-detection of cell membrane and nuclear membrane proteins using differentially labeled aptamers (e.g., aptamers with different fluorophores and barcode sequences). In some aspects, the cell membrane associated protein is E-cadherin, N-cadherin, or a Na⁺/K⁺-ATPase. In some aspects, the nuclear membrane protein is a nuclear pore complex protein.

What is claimed is:

1. A method of preparing a spatial proteome sequencing library from a biological sample, the method comprising:
(a) providing a surface comprising:
a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence common across oligonucleotides within a spatial feature and a unique molecular identifier sequence;
(b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a cleavage site;
(c) removing aptamers in the plurality of aptamers that did not associate with a protein in the biological sample;
(d) cleaving the plurality of aptamers to release (i) the target nucleotide sequence and (ii) the aptamer barcode nucleotide sequence, thereby resulting in association of the target nucleotide sequence with the capture nucleotide sequence, and
thereby preparing the spatial proteome sequencing library.

2. The method of claim 1, wherein the surface further comprises a blocker nucleic acid that is hybridized to at least a portion of the capture nucleotide sequence.

3. The method of claim 2, wherein the blocker nucleic acid is removed from the capture oligonucleotide after step (c).

4. The method of any one of claims 1-3, wherein the plurality of aptamers is cleaved via ultraviolet radiation, an enzyme, or chemical cleavage.

5. The method of any one of claims 1-4, further comprising (e) extending the capture nucleotide sequence to create copies of the individual aptamers, thereby creating extended capture oligonucleotides.

6. The method of claim 5, further comprising (f) adding a template switch oligonucleotide (TSO) to the 3′ end of the extended capture oligonucleotides.

7. The method of claim 6, wherein the TSO is directly ligated to the extended capture oligonucleotide.

8. A method of preparing a spatial proteome sequencing library from a biological sample, the method comprising:
(a) providing a surface comprising:
a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence common across oligonucleotides within a spatial feature and a unique molecular identifier sequence;
(b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; (ii) an aptamer barcode nucleotide sequence; and (iii) a truncated adapter nucleotide sequence;
(c) removing aptamers in the plurality of aptamers that did not associate with a protein in the biological sample;
(d) eluting the individual aptamers from the individual proteins, thereby resulting in association of the target nucleotide sequence with the capture nucleotide sequence, and
thereby preparing the spatial proteome sequencing library.

9. The method of claim 8, wherein the surface further comprises a blocker nucleic acid that is hybridized to at least a portion of the capture nucleotide sequence.

10. The method of claim 9, wherein the blocker nucleic acid is removed from the capture oligonucleotide after step (c).

11. The method of any one of claims 8-10, further comprising (e) extending the capture nucleotide sequence to create copies of the individual aptamers, thereby creating extended capture oligonucleotides.

12. The method of claim 11, further comprising (f) hybridizing the truncated adapter nucleotide sequence to a full length adapter nucleotide sequence primer and extending to synthesize a second strand.

13. A method of preparing a spatial proteome sequencing library from a biological sample, the method comprising:
(a) providing a surface comprising:
a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence common across oligonucleotides within a spatial feature and a unique molecular identifier sequence;
(b) contacting a plurality of aptamers to the biological sample on the surface, the contacting resulting in association of individual aptamer complexes in the plurality of aptamer complexes with individual proteins in the biological sample, wherein each aptamer complex in the plurality of aptamer complexes comprises:
(1) an aptamer comprising (i) the capture nucleotide sequence; and (ii) an aptamer-specific nucleotide sequence; and
(2) an oligonucleotide hybridized to the aptamer prior to the contacting, the oligonucleotide comprising (i) the target nucleotide sequence; (ii) a sequence complementary to the aptamer-specific nucleotide sequence; and (iii) an aptamer barcode nucleotide sequence,
wherein after the association of individual aptamer complexes in the plurality of aptamer complexes with individual proteins in the biological sample, the oligonucleotide is released from the aptamer thereby resulting in association of the target nucleotide sequence of the released oligonucleotide with the capture nucleotide sequence of a capture oligonucleotide of the plurality of capture oligonucleotides;
thereby preparing the spatial proteome sequencing library.

14. The method of claim 13, wherein the aptamer-specific nucleotide sequence is about 5 to about 20 nucleotides in length.

15. The method of claim 13, wherein the aptamer-specific nucleotide sequence is about 10 nucleotides in length.

16. The method of any one of claims 1-15, wherein the surface further comprises a blocker nucleic acid that is hybridized to at least a portion of the capture nucleotide sequence.

17. The method of claim 16, wherein the blocker nucleic acid is removed from the capture oligonucleotide after the contacting.

18. The method of any one of claims 13-17, wherein the association of individual aptamer complexes in the plurality of aptamer complexes with individual proteins in the biological sample results in release of the oligonucleotide from the aptamer.

19. The method of any one of claims 13-17, wherein after the association of individual aptamer complexes in the plurality of aptamer complexes with individual proteins in the biological sample, a condition is changed thereby resulting in release of the oligonucleotide from the aptamer.

20. The method of claim 19, wherein the condition is temperature, pH, or salt concentration.

21. The method of any one of claims 13-20, wherein after the association of individual aptamer complexes in the plurality of aptamer complexes with individual proteins in the biological sample, formamide is added thereby resulting in release of the oligonucleotide from the aptamer.

22. The method of any one of claim 3, 10, or 17, wherein the blocker oligonucleotide is removed from the capture oligonucleotide by exonuclease digestion.

23. The method of claim 22, wherein the exonuclease digestion is performed using T7 exonuclease or lambda exonuclease. 24 A method of preparing a spatial proteome sequencing library from a biological sample, the method comprising:
(a) providing a surface comprising:
a plurality of capture oligonucleotides immobilized on the surface, wherein each capture oligonucleotide in the plurality of capture oligonucleotides comprises (i) a capture nucleotide sequence at the 3′ end that is configured to bind to a target nucleotide sequence; and (ii) a unique molecular identifier (UMI) nucleotide sequence, wherein the UMI comprises a spatial barcode nucleotide sequence common across oligonucleotides within a spatial feature and a unique molecular identifier sequence;
(b) contacting a plurality of aptamers to the biological sample on the surface, the contacting resulting in association of individual aptamers in the plurality of aptamers with individual proteins in the biological sample, wherein each aptamer in the plurality of aptamers comprises (i) the target nucleotide sequence; and (ii) an aptamer barcode nucleotide sequence;
(c) removing aptamers in the plurality of aptamers that did not associate with a protein in the biological sample;
(d) digesting the proteins in the biological sample, thereby releasing the individual aptamers from the individual proteins, thereby resulting in hybridization of the target nucleotide sequence with the capture nucleotide sequence, and thereby preparing the spatial proteome sequencing library.

25. The method of claim 24, wherein the plurality of capture oligonucleotides comprises a cleavable site at the 5′ end.

26. The method of claim 24 or 25, wherein step (d) further comprises contacting at least one aptamer of the plurality of aptamers with a blocker nucleic acid, thereby forming a blocked aptamer, wherein the blocker nucleic acid is complementary to the target nucleotide sequence, and wherein the blocked aptamer is unable to associate with the capture nucleotide sequence.

27. The method of any one of claims 24-26, wherein the surface further comprises a blocker nucleic acid that is hybridized to at least a portion of the capture nucleotide sequence.

28. The method of claim 27, wherein the blocker nucleic acid is removed from the capture oligonucleotide after step (c).

29. The method of any one of claims 24-28, further comprising (e) extending the capture nucleotide sequence to create copies of the individual aptamers, thereby creating extended capture oligonucleotides.

30. The method of claim 29, wherein step (e) further comprises hybridizing a plurality of aptamer barcoded oligonucleotides to the extended capture oligonucleotides, and extending the extended capture oligonucleotides, thereby creating a plurality of barcoded capture oligonucleotides, wherein each of the aptamer barcoded oligonucleotides comprises at least a portion of an individual aptamer sequence.

31. The method of claim 30, wherein the plurality of aptamer barcoded oligonucleotides comprise a plurality of aptamer blocker nucleic acids, wherein each of the aptamer blocker nucleic acids comprises at least a portion of an individual aptamer sequence.

32. The method of any one of claims 24-31, further comprises cleaving the cleavable site, thereby releasing the plurality of capture oligonucleotides from the surface.

33. The method of any one of claims 1-32, wherein the aptamers comprise a detectable moiety.

34. The method of claim 33, wherein the detectable moiety is a fluorphore.

35. The method of any one of claims 1-34, wherein the method further comprises contacting a second plurality of aptamers to the biological sample on the surface, the contacting resulting in association of individual aptamers in the second plurality of aptamers with individual proteins in the biological sample, wherein each aptamer in the second plurality of aptamers comprises a detectable moiety.

36. The method of claim 35, wherein the detectable moiety is a fluorophore.

37. The method of claim 35 or 36, wherein each aptamer in the second plurality of aptamers comprises the target nucleotide sequence and a truncated adapter nucleotide sequence.

38. The method of claim 37, wherein each aptamer in the second plurality of aptamers further comprises an aptamer barcode nucleotide sequence.

39. The method of any one of claims 35-38, wherein after contacting the second plurality of aptamers to the biological sample on the surface, the method further comprises imaging the biological sample, thereby obtaining an image of the biological sample.

40. The method of claim 39, wherein the method does not comprise contacting the biological sample with hematoxylin and eosin (H&E) staining reagents.

41. The method of any one of claims 35-40, wherein at least one aptamer in the second plurality of aptamers is specific for a cell membrane-associated protein.

42. The method of any one of claims 35-40, wherein at least one aptamer in the second plurality of aptamers is specific for a nuclear membrane-associated protein.

43. The method of any one of claims 35-40, wherein at least one aptamer in the second plurality of aptamers is specific for a cell membrane-associated protein and at least one aptamer in the second plurality of aptamers is specific for a nuclear membrane-associated protein, and wherein the at least one aptamer specific for the nuclear membrane-associated protein comprises a different detectable moiety than the at least one aptamer specific for the cell membrane-associated protein.

44. The method of any one of claims 38-43, wherein the at least one aptamer specific for the nuclear membrane-associated protein comprises a different aptamer barcode nucleotide sequence than the at least one aptamer specific for the cell membrane-associated protein.

45. The method of any one of claims 41-44, wherein the cell membrane associated protein is E-cadherin, N-cadherin, or a Na⁺/K⁺-ATPase.

46. The method of any one of claims claim 42-44, wherein the nuclear membrane-associated protein is a nuclear pore complex protein.

47. The method of any one of claims 1-46, wherein the biological sample is from a mammal.

48. The method of any one of claims 1-47, wherein the biological sample is from a human.

49. A method of identifying a disorder in a subject having or at risk of having the disorder comprising:
i) generating a spatial proteomic and/or transcriptomic library from a biological sample from the subject according to the methods of the disclosure,
ii) comparing proteomic and/or genetic information from the sample proteomic and/or transcriptomic library to a control proteomic and/or transcriptomic library,
iii) identifying a genetic variation in the sample proteomic and/or transcriptomic library associated with the disease.

50. The method of claim 49, wherein the disorder is a neurodegenerative disorder.

51. The method of claim 49 or 50, wherein the disorder is Alzheimer's disease.
